(19) United States
(12) Patent Application Publication
(10) Pub. No.: US 2012/0270812 A1
(43) Pub. Date: Oct. 25, 2012

(54) COMPOSITIONS, METHODS, AND KITS FOR DETERMINING AN ALKYL TRANSFERASE

(75) Inventors: Michael R. Zalutsky, Durham, NC (US); Ganesan Vaidyanathan, Durham, NC (US)

(73) Assignee: Duke University, Durham, NC (US)

(21) Appl. No.: 13/381,677

(22) PCT Filed: Aug. 24, 2010
(86) PCT No.: PCT/US2010/046432

§ 371 Date: Jun. 22, 2012

Related U.S. Application Data
(60) Provisional application No. 61/236,334, filed on Aug. 24, 2009.

(51) Int. Cl. C07K 17/02; C12Q 1/68; G01N 33/573; G01N 27/26; A61K 38/08; A61P 35/00

(52) U.S. Cl. 514/21.7; 530/329; 435/188; 435/7.4; 435/6.13

(57) ABSTRACT

The present invention relates to novel compounds as well as to compositions, methods, and kits comprising the compounds for determining an alkyltransferase (ATase), in particular an alkylguanine-DNA alkyl transferase (AGT). In general, the novel compounds provide for determining ATase levels, in particular for in vivo applications including, but not limited to, theranostic applications, in particular to cancer-related applications.

### FIELD OF THE INVENTION

The present invention relates to compositions, methods, and kits for determining an alkyltransferase (ATase), in particular an alkylguanine-DNA alkyl transferase (AGT).

### BACKGROUND OF THE INVENTION

Many patients with various types of cancer receive chemotherapy as an important part of their treatment regimen and alkylating agents are one of the most common classes of chemotherapeutics. Alkylating agents are effective in some patients but ineffective in others. It has been suggested that the success or failure of chemotherapy in a particular patient largely depends on whether the patient's tumor has high or low levels of a DNA-alkyltransferase (ATase), namely O⁶-Alkylguanine DNA alkyltransferase (AGT; also known as O⁶-methylguanine-DNA methyltransferase (MGMT); EC2.1.1.64). This is because AGT is a DNA repair protein that can actually repair the damage done to the tumor by the chemotherapy, rendering it ineffective. For example, temozolomide is a chemotherapeutic that when combined with radiation therapy, can improve the survival of patients with brain tumors. However, this is only the case if patients do not have high levels of AGT.

Several preclinical and clinical studies have established an inverse correlation between survival and AGT levels in a tumor. These studies have suggested that it is futile to administer chemotherapeutic agents if the tumor to be treated has AGT in amounts considerably higher than a threshold level.

Ex vivo methods are available to determine AGT content, however, these are performed on tumor samples obtained by biopsies. There are a number of problems with this: 1) it is invasive; 2) tumor may be located where biopsy is not possible; 3) results may not reflect the tumor as a whole because a biopsy samples only a small region of the tumor, which can be heterogeneous in their behavior; and 4) biopsy approach is not suitable for following patients over time, to monitor their progress after treatment has begun and fine tune the treatment.

Accordingly, there is a need for effective compounds, compositions, methods, and kits for determining an ATase.

### SUMMARY OF THE INVENTION

In one aspect, the present invention provides a compound comprising a substrate for an ATase, wherein the substrate is coupled to a polypeptide. In some embodiments, the substrate is an O⁶-benzylguanine (BG).

In another aspect, the present invention provides a compound having the formula (I):

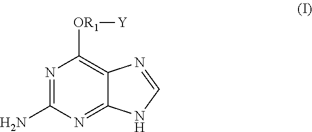

wherein R₁ is a benzyl group, wherein Y is a polypeptide.

In other aspects, the present invention provides a compound having the formula (I):

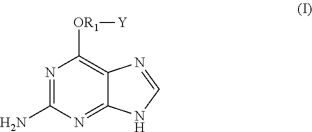

wherein R₁ is a benzyl group substituted at the ortho, meta, or para position with:

an azide functional group,

an azido-hexyloxymethyl group,

R₂R₃ where R₂ represents an alkyl of 1-4 carbon atoms and R₃ represents an azide functional group or an azido-hexyloxymethyl group,

R₄R₅ where R₄ represents carbonyl and R₅ represents succinimidyloxy, or

R₆R₇R8 where R₆ represents a hexyloxymethyl group, R₇ represents an amine, and R8 represents a cyclooctyne group; and

wherein Y is a polypeptide.

In some aspects, the present invention provides a compound having the formula (II):

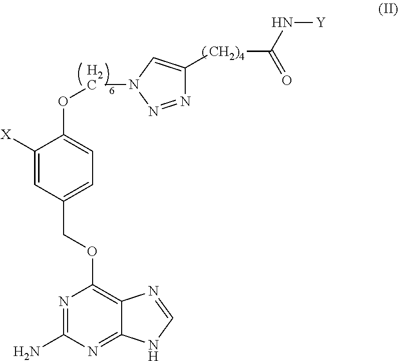

wherein X is a halogen atom, a radiohalogen, or a radiometal complexed to a chelating group, wherein Y is a polypeptide. In one aspect, the present invention provides a compound having the formula (III):

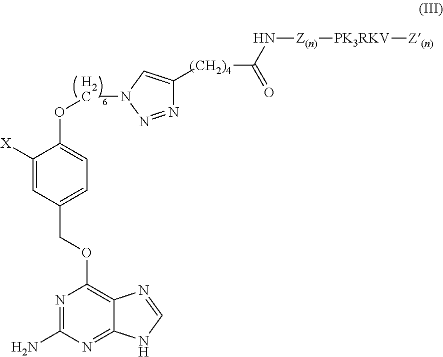

wherein X is a halogen atom, a radiohalogen, or a radiometal complexed to a chelating group, wherein Z and Z′ are each independently an amino acid, wherein n is an integer greater than or equal to zero.

In another aspect, the present invention provides a compound comprising a substrate for an ATase, wherein the substrate comprises a reporting group capable of undergoing a reaction with a probe having a labeled group to provide a labeled substrate.

In other aspects, the present invention provides a compound having the formula (IV):

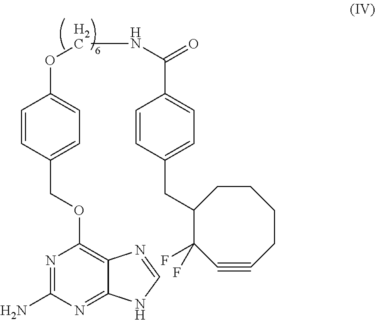

In some aspects, the present invention provides a method for preparing a compound comprising a substrate for an ATase, the method comprising:

(a) performing a click reaction between an O⁶-benzylguanine (BG) having an azide functional group with a polypeptide having an alkyne functional group whereby the substrate is coupled to a polypeptide.

In another aspect, the present invention provides a method for preparing a compound comprising a substrate for an ATase, the method comprising:

(a) conjugating an O⁶-benzylguanine (BG) having an active ester group with a polypeptide having an amine functional group, wherein the active ester group reacts with the amine functional group to form an amide linkage.

In other aspects, the present invention provides a composition comprising a compound of the present invention. In some embodiments, the composition further comprises a pharmaceutically acceptable carrier.

In one aspect, the present invention provides a method for labeling an ATase, the method comprising:

contacting a compound with the ATase, wherein the compound comprises a substrate for the ATase, wherein the substrate is coupled to a polypeptide, wherein the substrate is labeled with a detectable label bound to a chemical substituent of the substrate.

In another aspect, the present invention provides a method of detecting an ATase in a subject, the method comprising:

(a) contacting the AGT of the subject with an O⁶-derivatized guanine compound comprising at the exocyclic O⁶ position a radiolabeled alkyl or benzyl group covalently coupled to a polypeptide under conditions whereby the radiolabeled alkyl or benzyl group is transferred from the O⁶-derivatized guanine compound to the AGT to form a radiolabeled AGT molecule; and

(b) detecting the radiolabeled AGT molecule.

In other aspects, the present invention provides a method for in vivo labeling an ATase in a subject, the method comprising:

administering to the subject a non-labeled substrate for an ATase, wherein the substrate has a reporting group that is bioorthogonal to a group of a labeled probe.

In some aspects, the present invention provides a method for determining a treatment regimen for a subject, the method comprising:

determining the subject's ATase levels, wherein determining comprises contacting an ATase of the subject with a compound comprising a substrate for an ATase, wherein the substrate is coupled to a polypeptide, wherein the substrate is labeled with a detectable label bound to a chemical substituent of the substrate, wherein the subject's ATase levels determine the treatment regimen.

In one aspect, the present invention provides a method for determining a treatment regimen for a subject, the method comprising

administering to the subject a non-labeled substrate for an ATase, wherein the substrate has a reporting group that is bioorthogonal to a group of a labeled probe.

In some aspects, the present invention provides a method for monitoring the effect of a reagent on the amount of AGT molecules in a tumor in a subject, the method comprising:

determining the amount of AGT molecules in the tumor before, after, or contemporaneously with administration of the reagent, wherein determining comprises:

(a) contacting the AGT of the subject with an O⁶-derivatized guanine compound comprising at the exocyclic O⁶ position a radiolabeled alkyl or benzyl group covalently coupled to a polypeptide under conditions whereby the radiolabeled alkyl or benzyl group is transferred from the O⁶-derivatized guanine compound to the AGT to form a radiolabeled AGT molecule; and

(b) detecting the amount of radiolabeled AGT molecules in the tumor relative to a control in which no reagent is administered.

In one aspect, the present invention provides a method for determining the efficacy of a subject's treatment, the method comprising:

administering to the subject a non-labeled substrate for an ATase, wherein the substrate has a reporting group that is bioorthogonal to a group of a labeled probe.

In another aspect, the present invention provides a method for screening for a molecule to identify candidate molecules that reduce or inhibit the expression and/or biological function/activity of an ATase, the method comprising:

determining a subject's ATase levels, wherein the subject is administered a candidate molecule, wherein determining comprises contacting an ATase of the subject with a compound comprising a substrate for an ATase, wherein the substrate is coupled to a polypeptide, wherein the substrate is labeled with a detectable label bound to a chemical substituent of the substrate, wherein ATase levels are indicative of reduction or inhibition of expression and/or biological function/activity of the ATase by the candidate molecule.

In other aspects, the present invention provides a method for screening for a molecule to identify candidate molecules that reduce or inhibit the expression and/or biological function/activity of an ATase of a subject, the method comprising:

administering to the subject a non-labeled substrate for an ATase, wherein the substrate has a reporting group that is bioorthogonal to a group of a labeled probe.

In still further aspects, the present invention provides use of a compound of the present invention for the preparation of a composition suitable for administration to a subject for targeted imaging and screening.

In other aspects, a kit is provided, wherein the kit comprises a compound and/or composition in accordance with the present invention.

### BRIEF DESCRIPTION OF THE DRAWINGS

FIG. 1 is a schematic depicting one embodiment of preparation of SEM-protected O⁶-(4-Azidohexyloxymethyl-3-iodo)benzylguanine (AHOMIBG) and its tin precursor.

FIG. 2 is a schematic depicting one embodiment of preparation of AHOMIBG conjugated with PK₃RKV (SEQ ID NO:1).

FIG. 3 is a schematic depicting one embodiment of preparation of [¹³¹I]CIBG-NHS.

FIG. 4 is a schematic depicting one embodiment of preparation of a BG derivative appended with a cyclooctyne group.

FIG. 5 is a schematic depicting preparation of ¹⁸F-labeled compound 25 and coupling to the guanine skeleton.

FIG. 6 is a schematic depicting preparation of compound 7 from compound 4 and commercially available 3-iodobenzyl alcohol in 60% isolated yield and converted to compound 8 by treatment with sodium hydride or potassium tert-butoxide, and SEM-Cl.

FIG. 7 depicts various examples of compounds in accordance with the present invention.

FIG. 8 is a graph showing depletion of cellular AGT activity by unlabeled 6-(4-fluoro-benzyloxy)-9H-purin-2-ylamine (O⁶-4-fluorobenzylguanine (FBG)) and 6-(iodo-benzyloxy)-9H-purin-2-ylamine (O⁶-iodobenzylguanine (IBG)). CHO cells transfected with pCMV-AGT were incubated with varying concentrations of IBG (▪) or FBG () for 4 hours, and the AGT activity associated with the cells was determined. The results are expressed as the percentage of the AGT activity present in cell cultures that were not treated with FBG or IBG.

FIG. 9 is a graph showing binding of [¹⁸F] FBG to purified AGT as a function of unlabeled FBG concentration. [¹⁸F] FBG was incubated for 30 minutes at 37° C., in the presence or absence of increasing amounts of unlabeled FBG, with 10 μg of AGT (), or to control for nonspecific binding, 10 μg of BSA (▴) in a Tris-buffer. The protein-associated activity was determined by TCA precipitation.

FIG. 10 is a graph showing binding of [¹³¹I] IBG to purified AGT as a function of unlabeled IBG concentration. The assay was performed as in FIG. 9 by incubating [¹³¹I] IBG with AGT () or BSA (▴).

### DETAILED DESCRIPTION

There is now provided novel compounds and uses thereof as novel substrates for an ATase. The compounds can serve as the basis for determining the ATase. The novel compounds of the present invention also can serve as the basis for a variety of applications and methods including, but not limited to, theranostic and diagnostic applications relating to ATase expression/activity, in particular as it relates to cancer.

### I. Compound

In one aspect, the present invention provides a compound comprising a substrate for an ATase, wherein the substrate is coupled to a polypeptide.

The ATase can be any ATase protein or a derivative thereof, either naturally or recombinantly expressed. ATase variability and regulation is described in, e.g., Margison et al., Carcinogenesis, 24:625 (2003), which is herein incorporated by reference for its teaching of ATases and corresponding Genbank accession numbers.

In some embodiments, the ATase is human AGT or a derivative thereof.

### A. Substrate

Generally, the substrate has a chemical substituent that can be transferred to an active-site amino acid residue (e.g., active-site cysteine) of the ATase upon contact of the substrate with the ATase. In some embodiments, the transfer of the chemical substituent is a stoichiometric transfer of the chemical substituent and is associated with inactivation of the ATase.

In one embodiment, the substrate is a purine or a pyrimidine analogue. For example, the purine analogue can be, but is not limited to, a guanine comprising the chemical substituent (e.g., a benzyl group or moiety) attached thereto at the O(6)-position of the guanine. For example, the substrate can be O⁶-benzylguanine (BG). Or, for example, the pyrimidine analogue can be, but is not limited to, a thymine having the chemical substituent attached thereto at the O(4)-position of thymine. Non-limiting examples of ATase substrates are disclosed by, e.g., U.S. Pat. Nos. 5,091,430; 5,352,669; 5,358,952; 5,525,606; 5,691,307; 5,753,668; 5,916,894; 5,958,932; 6,172,070; 6,303,604; 6,333,331; and 6,436,945; U.S. Patent Publication Nos. 2007/0243568 and 2006/0024775; Ciocco et al., Cancer Res. 55:4085-91 (1995); Chae et al., J Med Chem., 37(3):342-7 (1994); Dolan et al., PNAS, 87:5368-5372 (1990); Hotta et al., J Neurooncol., 21(2):135-40 (1994); Moschel et al., J Med Chem., 35:4486-91 (1992); and Mounetou et al., J. Labeled Compounds and Radiopharmaceuticals, 36: 1215-1225 (1995), each of which is herein incorporated by reference for its teaching of ATase substrates.

The terms “group” and “moiety,” as used herein, are intended to distinguish between chemical species that allow for substitution or that may be substituted and those that do not allow or may not be so substituted. Thus, when the term “group” is used to describe a chemical substituent, the described chemical material includes the unsubstituted group and that group with O, N, or S atoms, for example, in the chain as well as carbonyl groups or other conventional substitution. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as phenyl, hydroxy, alkoxy, alkylsulfonyl, halogen atoms, cyano, nitro, amino, carboxyl, etc. Thus, “alkyl group” includes aralkyls, ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, t-butyl, and the like.

In other embodiments, the substrate is part of a polynucleotide comprising the substrate, wherein the substrate is coupled to the polypeptide, either directly or indirectly via a nucleotide of the polynucleotide. In some embodiments, the polynucleotide comprises at least 1, illustratively, at least 1, at least 2, at least 3, at least 4, at least 5, at least 10, at least 20, at least 30, at least 50, or more nucleotides other than the substrate. U.S. Pat. No. 6,060,458 is herein incorporated by reference for its teaching of polynucleotides comprising O⁶-benzylguanine.

In one embodiment, the substrate is a guanine having the chemical substituent attached thereto at the 0(6)-position. In another embodiment, the chemical substituent is a group or moiety selected from the group consisting of benzyl-, p-chlorobenzyl-, and p-methylbenzyl.

In some embodiments, the substrate is O⁶-benzylguanine (BG), wherein the substrate is coupled to a polypeptide.

In one embodiment, the compound has the formula (I):

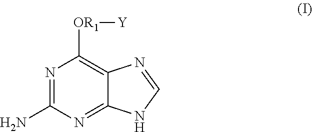

wherein R₁ is a benzyl group, wherein Y is a polypeptide. In some embodiments, R₁ is a substituted at the ortho, meta, or para position with a halogen atom or an radioisotope thereof. In another embodiment, optionally, a linker group or moiety couples R₁ with Y.

In some embodiment, the compound has the formula (I), wherein R₁ is a benzyl group substituted at the ortho, meta, or para position with:

an azide functional group,

an azido-hexyloxymethyl group,

R₂R₃ where R₂ represents an alkyl of 1-4 carbon atoms and R₃ represents an azide functional group or an azido-hexyloxymethyl group,

R₄R₅ where R₄ represents carbonyl and R₅ represents succinimidyloxy, or

R₆R₇R8 where R₆ represents a hexyloxymethyl group, R₇ represents an amine, and R8 represents a cyclooctyne group; and

wherein Y is a polypeptide.

In some embodiments, R₁ is further substituted at the ortho, meta, or para position with a halogen atom or an radioisotope thereof.

In another embodiment, the compound has the formula (II):

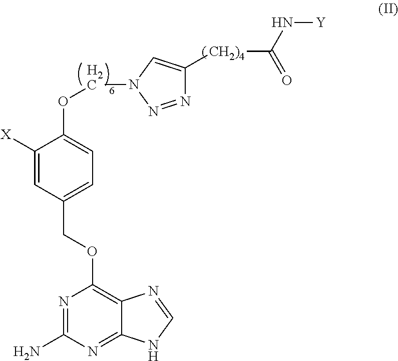

wherein X is a halogen atom, a radiohalogen, or a radiometal complexed to a chelating group, wherein Y is a polypeptide.

In some embodiments, Y comprises the amino acid sequence: proline-lysine-lysine-lysine-arginine-lysine-valine (PK₃RKV) (SEQ ID NO:1).

In other embodiments, the compound has the formula (III):

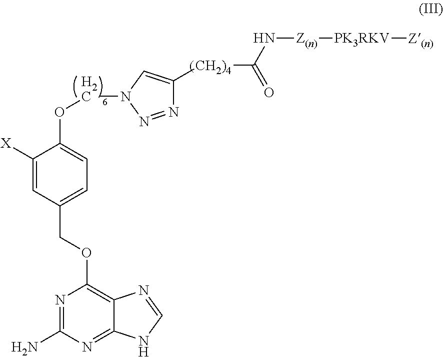

wherein X is a halogen atom, a radiohalogen, or a radiometal complexed to a chelating group, wherein Z and Z′ are each independently an amino acid, wherein n is an integer greater than or equal to zero.

### B. Polypeptide

The polypeptide that is coupled to the substrate can be any polypeptide, preferably a polypeptide comprising an amino acid sequence that confers a functional property to the substrate. The term “functional property,” as used herein, is intended to be broad and includes a property that the substrate does not possess in the absence of the polypeptide being coupled thereto, or a property that the substrate possesses but which is effected (e.g., enhanced, diminished) by virtue of the polypeptide being coupled thereto.

For example, the functional property can include, but is not limited to, a targeting (e.g., nuclear localization, cell-specificity) feature for targeting the compound having the substrate coupled to the polypeptide; an interacting (e.g., binding) feature for interacting the compound with other molecules (e.g., accessory proteins, receptors, nucleic acids); a cell penetrating feature for cellular uptake of the compound; an endosome escape feature (e.g., an endosmolytic related component that enables escape of the compound from the endosome; a purifying feature (e.g. His-tags, biotin) for purification of the compound; a detecting feature (e.g., a carrier polypeptide for attaching a detection label (e.g., fluorescent label (e.g., fluorescein, CY3, Cy5), radioactive label); structural features (e.g., spacer (e.g., Gly-Ser)₅), protease-cleavable linker, zinc finger, etc.); catalytic features; and combinations thereof.

In other embodiments, the amino acid sequence of the polypeptide can, but need not, confer more than one functional property to the substrate having the polypeptide coupled thereto. For example, the polypeptide that is coupled to the substrate can comprise an amino acid sequence (e.g., NLS, α-melanocyte stimulating hormone (MSH), EGF, and fragments thereof) that confers a cell internalizing property and/or a cell-specific targeting property to the compound. By way of another example, the amino acid sequence can be used to detect and/or purify the compound.

In one embodiment, the functional property is a targeting property whereby the compound is targeted to a cell (e.g., tumor cells, liver cells, haematopoietic cells, etc.) or a cellular compartment (e.g., nucleus, mitochondria). For example, in various embodiments, the targeting amino acid sequence can be, but is not limited to, all or a portion of a nuclear localization sequence (NLS), a hormone (e.g., MSH, insulin), a growth factor (e.g., EGF), a cell receptor, a cytokine, a glycoprotein (e.g., transferrin, thrombomodulin), an antibody, a fusogenic agent (e.g., polymixin B, hemagglutinin HA2), etc., including functional variants thereof. In other embodiments, the polypeptide itself can be further coupled (e.g., via amino groups) to a targeting ligand such as, for example, a carbohydrate. Suitable carbohydrates/sugars can include mono- or oligo-saccharides, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid. For example, the galactosyl unit of lactose can provide targeting to a hepatocyte having a galactose receptor. Other examples of a targeting ligand include, but are not limited to, asialoorosomucoid, Lewis^{X}, and sialyl Lewis^{X}.

### 1. NLS

In some embodiments, the polypeptide comprises an amino acid sequence corresponding to a nuclear localization sequence (NLS). A variety of NLSs are known in the art and include, but are not limited to, the NLS of the SV40 virus large T-antigen, wherein the minimal functional unit is the seven amino acid sequence PKKKRKV (SEQ ID NO: 1). Other non-limiting examples of NLSs include the nucleoplasmin-based bipartite sequence NLSKRPAAIKKAGQAKKKK (SEQ ID NO: 2), the c-myc-based sequences PAAKRVKLD (SEQ ID NO: 3) and/or RQRRNELKRSF (SEQ ID NO: 4), the hRNPA1 M9-based sequence NQSSNFGPMKGGNFGGRSSGPYGGGGQYFAKPRNQGGY (SEQ ID NO: 5), the importin-alpha IBB domain-based sequence RMRKFKNKGKDTAELRRRRVEVSVELRKAKKDEQILKRRNV (SEQ ID NO: 6), the myoma T protein-based sequences VSRKRPRP (SEQ ID NO: 7) and/or PPKKARED (SEQ ID NO: 8), the human p53-based sequence PQPKKKPL (SEQ ID NO: 9), the mouse c-abl IV-based sequence SALIKKKKKMAP (SEQ ID NO: 10), the sequences DRLRR (SEQ ID NO: 11) and PKQKKRK (SEQ ID NO: 12) of the influenza virus NS1, the sequence RKLKKKIKKL (SEQ ID NO: 13) of the Hepatitis virus delta antigen, and the mouse Mx1-based sequence REKKKFLKRR (SEQ ID NO: 14), the human poly(ADP-ribose) polymerase-based bipartite sequence KRKGDEVDGVDEVAKKKSKK (SEQ ID NO: 15), and the steroid hormone receptor-based sequence RKCLQAGMNLEARKTKK (SEQ ID NO: 16), and combinations thereof.

In one embodiment, the polypeptide comprises at least two NLSs, wherein the NLSs comprise the same or a different amino acid sequence. In some embodiments, the polypeptide comprises the amino acid sequence PKKKRKV (SEQ ID NO: 1). In other embodiments, the polypeptide comprises the amino acid sequence corresponding to a dimer, a trimer, or a tetramer of the amino acid sequence PKKKRKV (SEQ ID NO: 1).

### 2. Cell-Penetrating/Internalizing Sequence

In other embodiments, the polypeptide that is coupled to the substrate comprises an amino acid sequence corresponding to a cell-penetrating peptide (CPP). For example, the amino acid sequence can be a polycationic sequence (e.g., at least about 5 consecutive arginine residues). In another embodiment, the CPP is fused to a further amino acid sequence corresponding to an inhibitory domain made up of negatively charged residues thereby providing an activatable CPP (ACPP). In some embodiment, the ACPP comprises a linker sequence between the polycationic and polyanionic domains, wherein the linker is cleavable, e.g. by a protease or reduction of a disulfide bond, to release the CPP portion to bind to and enter cells, wherein the CPP potion is coupled to the substrate. Thus, in various other embodiments, the polypeptide comprises an amino acid sequence characterized as an activatable CPP (ACPP) and, optionally, a cleavable linker. CPPs and ACPPs are described by, e.g., Jiang et al., PNAS, 101:17867-17872 (2004) and U.S. Publication No. 2007/0041904, each of which is herein incorporated by reference for its teaching of CPPs and ACPPs.

In one embodiment, the polypeptide comprises an amino acid sequence selected from the group consisting of
EEEEEDDDDKAXRRRRRRRRRXC (SEQ ID NO:17);
EEEEEDDDDKARRRRRRRRRXC (SEQ ID NO:18);
EDDDDKAXRRRRRRRRRXC (SEQ ID NO:19);
EEDDDDKARXRRXRRXRRXRRXC (SEQ ID NO:20); and
DDDDDDKARRRRRRRRRXC (SEQ ID NO:21), wherein X corresponds to 6-aminohexanoic acid (aminocaproic acid). In some embodiments, the polypeptide further comprises a PEG tail.

In still other embodiments, the polypeptide comprises a sequence corresponding to at least a segment of a cell-specific ligand. In one embodiment, the cell-specific ligand is MSH, EGF, insulin-like growth factor, nerve growth factor, or somatostatin.

In another embodiment, the present invention provides a compound comprising a substrate for an ATase, wherein the substrate is coupled to a polypeptide, wherein the ATase is an O⁶-alkylguanine DNA alkyltransferase, wherein the polypeptide comprises an amino acid sequence shown as SEQ ID NO:1, wherein the polypeptide, optionally, further comprises a second amino acid sequence selected from the group consisting of: (SEQ ID NO:17); (SEQ ID NO:18); (SEQ ID NO:19); (SEQ ID NO:20); and (SEQ ID NO:21). In one embodiment, the amino acid sequence and the second amino acid sequence are separated by one or more amino acid residues.

### 3. Modular Recombinant Transporter (MRT)

In other embodiments, the polypeptide comprises an amino acid sequence corresponding to an MRT for targeting a cancer cell. MRTs are described by, e.g., Gilyazova et al., Cancer Res., 66:10534-40 (2006), which is herein incorporated by reference for its teaching of MRTs and targeting cancer cells.

### C. Coupled

The term “coupled,” as used herein in the context of the substrate being “coupled” to the polypeptide, includes covalent and non-covalent interactions, preferably covalent.

For example, the substrate can be modified with an azide function and the polypeptide can have an alkyne function whereby the substrate and the polypeptide can be conjugated via a click reaction. A click reaction is described by, e.g., Lutz et al., Adv. Drug Deliv. Rev., 60:958-70 (2008), which is herein incorporated by reference for its teaching of azide-alkyne click chemistry. Thus, for example, a substrate derivative appended with an azido-hexyloxymethyl group can be synthesized and conjugated to a heptynoyl-modified polypeptide comprising an amino acid sequence corresponding to an NLS, for example an NLS derived from SV40 T-antigen (e.g., SEQ ID NO:1)).

By way of example, in another embodiment, substrate derivatives comprising an active ester group can be coupled to amine functions in the polypeptide.

In some embodiments, the polypeptide is coupled to the chemical substituent of the substrate. For example, wherein the substrate is a guanine, the polypeptide can be coupled to an exocyclic position of the guanine, preferably to the group or moiety at the O⁶-position of the guanine, for example to a benzyl group or moiety.

### D. Label

In other embodiments, at least the substrate portion of the compound is labeled, preferably with a detectable label covalently bound to the chemical substituent of the substrate, either directly or through a linker. Radiolabeled substrates for an ATase are described by, e.g., International Publication No. WO 01/85221, which is herein incorporated by reference in its entirety. In one embodiment, the label is a radiolabel. In some embodiments, the label can emit or be caused to emit detectable radiation (e.g. by radioactive decay). Thus, in some embodiments, the label (e.g., a radiolabel) transfers to an active-site amino acid residue of an ATase upon contact of at least the substrate portion of the compound with the ATase.

Suitable radiolabels include, but are not limited to, 125I, ¹²³I, ¹²⁴I, ¹⁸F, ⁷⁵Br, ⁷⁶Br, ⁷⁷Br, and ¹¹C. In some embodiments, other elements and isotopes, may be applied for imaging including radiometals such as ¹¹¹In, complexed to a chelating group (e.g., 1,4,7,10-tetraazacyclododecane-1,4,7,10-tetraacetic acid (DOTA). Thus, other elements, isotopes, and imagining agents not specifically recited herein also may be used as labels.

For example, in some embodiments, the present invention provides iodinated O⁶-benzylguanine-derivatives. Iodine has a spectrum of radionuclides with different physical properties that can be suitable for a variety of applications including, but not limited to, imaging. For example, ¹²³I (t_{1/2}=13 hours) is a radionuclide that has been utilized in single photon emission tomography (SPECT). ¹²⁴Iodine (t_{1/2}=4.2 days) has been used for PET.

For example, in other embodiments, a non-radiolabeled trimethyl tin precursor can be prepared from a parent molecule having an iodo group by replacing the iodo group of the parent with a trimethyl tin group in the presence of bis(trimethyl)tin ((Me₃Sn)₂) and dichlorobis(triphenylphosphine)palladium (II) ((Ph₃P)₂PdCl₂). The radiolabeled substrate can then be prepared from the non-radiolabeled trimethyl tin precursor by oxidation with labeled sodium iodide, for example. Vaidyanathan et al., Bioconjug Chem. 11:868-875 (2000) is herein incorporated by reference for its teaching of preparing a radiolabeled guanine derivative.

For example, in one embodiment, isotopes such as ¹¹¹In, ¹²⁵I, ¹²³I, ¹²⁴I, ¹⁸F, ₇₆Br, ⁷⁶Br, ⁷⁷Br, and ¹¹C can be used to provide a radiolabeled O⁶-derivatized guanine molecule. Preferably, the radiolabel resides within the chemical substituent (e.g., an alkyl or a benzyl group) attached to the exocyclic O⁶-position of the guanine.

In other embodiments, the detectable label is an imaging agent or a fluorescent molecule.

Suitable imaging agents include positive contrast agents and negative contrast agents. Suitable positive contrast agents include, but are not limited to, gadolinium tetraazacyclododecanetetraacetic acid (Gd-DOTA); Gadolinium-diethylenetriaminepentaacetic acid (Gd-DTPA); Gadolinium-1,4,7-tris(carbonylmethyl)-10-(2′-hydroxypropyl)-1,4,7,10-tetr-aazacyclododecane (Gd-HP-DO3A); Manganese(II)-dipyridoxal diphosphate (Mn-DPDP); Gd-diethylenetriaminepentaacetate-bis(methylamide) (Gd-DTPA-BMA); and the like. Suitable negative contrast agents include, but are not limited to, a superparamagnetic iron oxide (SPIO) imaging agent; and a perfluorocarbon, where suitable perfluorocarbons include, but are not limited to, fluoroheptanes, fluorocycloheptanes, fluoromethylcycloheptanes, fluorohexanes, fluorocyclohexanes, fluoropentanes, fluorocyclopentanes, fluoromethylcyclopentanes, fluorodimethylcyclopentanes, fluoromethylcyclobutanes, fluorodimethylcyclobutanes, fluorotrimethylcyclobutanes, fluorobutanes, fluorocyclobutanse, fluoropropanes, fluoroethers, fluoropolyethers, fluorotriethylamines, perfluorohexanes, perfluoropentanes, perfluorobutanes, perfluoropropanes, sulfur hexafluoride, and the like.

Suitable fluorescent molecules (fluorophores) include, but are not limited to, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethylrhodamine-, methyl ester), TMRE (tetramethylrhodamine, ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; 181446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, and fluorescent europium and terbium complexes.

In still further embodiments, an O⁶-derivatized guanine molecule useful as a substrate in the present invention comprises at the exocyclic O⁶ position a benzyl group or an alkyl group, such as an ethyl, n-propyl, or n-butyl group. Preferred moieties include fluoromethyl, fluoroethyl, fluoro-n-propyl, fluoro-n-butyl, ortho-fluoromethylbenzyl, ortho-fluoroethylbenzyl, ortho-fluoropropylbenzyl, meta-fluoromethylbenzyl, meta-fluoroethylbenzyl, meta-fluoropropylbenzyl, para-fluoromethylbenzyl, para-fluoroethylbenzyl, or para-fluoropropylbenzyl. In one embodiment, the O⁶-derivatized guanine molecule is a radiolabeled O⁶-benzylguanine molecule. A variety of substituents are tolerated in the benzene ring of O⁶-benzylguanine. For example, FBG ([6-(4-fluoro-benzyloxy)-9H-purin-2-ylamine; O⁶-4-fluorobenzylguanine]) is among the purine and pyrimidine derivatives that have been shown to be AGT depletors. The ability of FBG to deplete AGT in HT29 cell-free extracts and intact cells has been shown to be similar to that of O⁶-benzylguanine itself. In some embodiments, the substrate is a radiolabeled FBG, for example ¹⁸F-labeled FBG.

### II. Bioorthogonal

In other aspects, the present invention provides one or more reagents for labeling an ATase (e.g., an AGT) utilizing a pre-targeting strategy based, e.g., on activity-based protein profiling. In some embodiments, the present invention provides a non-labeled substrate (e.g., a non-labeled BG derivative) for an ATase, wherein the non-labeled substrate has a reporting group. In other embodiments, the present invention further provides a labeled probe comprising a group that is bioorthogonal to the reporting group of the non-labeled substrate. For example, a technique used for the conjugation of a bioorthogonal group can be strain-promoted cycloaddition for in vitro or in vivo conjugation. In one embodiment, BG derivatives comprising a cyclooctyne group or group can be used to target an ATase (e.g., AGT) in cells (e.g., tumor cells), wherein such a BG derivative can be utilized in tandem with a complementary azide function-comprising labeled probe that can form a triazole with the cyclooctyne group on the BG analogue at physiological conditions via strain-promoted cycloaddition. A labeled probe molecule (e.g., a labeled reverse transcriptase inhibitor (e.g., a radioiodinated AZT, ¹⁸F-labeled AZT)) that can localize in the cell nuclei can be used, for example. In other embodiments, labeled analogues of a nuclear staining molecule (e.g., labeled DAPI) can be utilized.

In one embodiment, the present invention provides a non-labeled substrate for an ATase, wherein the substrate comprises an alkyne group capable of reacting with an azide group of a probe molecule. In other embodiments, the substrate comprises an azide group capable of reacting with an alkyne group of a probe molecule. In some embodiments, the probe molecule is labeled with a detectable label.

In some embodiments, the alkyne group is an activated alkyne capable of undergoing a catalyst free [3+2] cycloaddition reaction with an azide group. In one embodiment, the alkyne group is a cycloalkyne, preferably a strained cycloalkyne. The strained cycloalkyne can, in some embodiments, be a heterocycloalkyne, e.g., the cycloalkyne can comprise atoms other than carbon. In other embodiments, the cycloalkyne or heterocycloalkyne is a 7-membered, an 8-membered, or a 9-membered ring. The strain on the cycloalkyne can be increased in a variety of ways, e.g., through the use of heteroatoms; the degree of unsaturation, or torsional strain; the use of electron-withdrawing groups, etc. U.S. Patent Publication No. 2007/0249014 to Agnew et al. and U.S. Patent Publication No. 2006/0110782 to Bertozzi et al. each is herein incorporated by reference for its teaching of compositions and methods for generating covalently modified molecules using orthogonal reactivity.

In some embodiments, the cycloalkyne is a cyclooctyne. In other embodiments, one or more of the carbon atoms in the cyclooctyne ring, other than the two carbon atoms joined by a triple bond, is substituted with one or more electron-withdrawing groups, e.g., a halo (e.g., bromo, chloro, fluoro, iodo), a nitro group, a cyano group, a sulfone group, or a sulfonic group.

In other embodiments, the non-labeled substrate has the formula (IV):

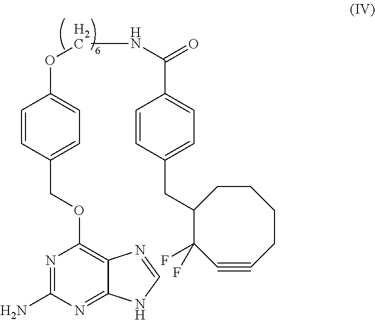

In one embodiment, the probe molecule further comprises a detectable label, covalently bound thereto either directly or through a linker.

Exemplary detectable labels include, but are not limited to, radioactive labels, imaging reagents, fluorescent molecules, and the like. In some embodiments, the label can emit or be caused to emit detectable radiation (e.g. by radioactive decay).

Suitable radioactive labels include, but are not limited to, ¹¹¹In, ¹²⁵I, ¹²³I, ¹²⁴I, ¹⁸F, ⁷⁵Br, ⁷⁶Br, ⁷⁷Br, and ¹¹C.

Suitable imaging agents include positive contrast agents and negative contrast agents. Suitable positive contrast agents include, but are not limited to, gadolinium tetraazacyclododecanetetraacetic acid (Gd-DOTA); Gadolinium-diethylenetriaminepentaacetic acid (Gd-DTPA); Gadolinium-1,4,7-tris(carbonylmethyl)-10-(2′-hydroxypropyl)-1,4,7,10-tetr-aazacyclododecane (Gd-HP-DO3A); Manganese(II)-dipyridoxal diphosphate (Mn-DPDP); Gd-diethylenetriaminepentaacetate-bis(methylamide) (Gd-DTPA-BMA); and the like. Suitable negative contrast agents include, but are not limited to, a superparamagnetic iron oxide (SPIO) imaging agent; and a perfluorocarbon, where suitable perfluorocarbons include, but are not limited to, fluoroheptanes, fluorocycloheptanes, fluoromethylcycloheptanes, fluorohexanes, fluorocyclohexanes, fluoropentanes, fluorocyclopentanes, fluoromethylcyclopentanes, fluorodimethylcyclopentanes, fluoromethylcyclobutanes, fluorodimethylcyclobutanes, fluorotrimethylcyclobutanes, fluorobutanes, fluorocyclobutanse, fluoropropanes, fluoroethers, fluoropolyethers, fluorotriethylamines, perfluorohexanes, perfluoropentanes, perfluorobutanes, perfluoropropanes, sulfur hexafluoride, and the like.

Suitable fluorescent molecules (fluorophores) include, but are not limited to, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethylrhodamine-, methyl ester), TMRE (tetramethylrhodamine, ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5”-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl)aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, and fluorescent europium and terbium complexes.

In another embodiment, the present invention provides a substrate for an AGT, wherein the substrate comprises a phosphine group that can be transferred to an active-site amino acid residue (e.g., an active-site cysteine) of the AGT upon contact of the substrate with the AGT, wherein the phosphine group can react in vitro, ex vivo, and/or in vivo, preferably in vivo, with an azide group of a radiolabeled probe comprising the azide group. For example, the phosphine group and the azide group can react in vivo by way of the Staudinger ligation, e.g., as described by U.S. Publication No. 2008/0274057 to Robillard et al. and International Publication No. WO/2006/021553 to Kindermann et al., Bertozzi, PNAS, 103:4819 (2006), Kiick et al., PNAS, 99:19 (2002), Vugts et al., Journal of Labeled Compounds and Radiopharmaceuticals, 52: S51 (2009); Vulders et al., Journal of Labeled Compounds and Radiopharmaceuticals, 52: S461 (2009); and Mamat et al., Journal of Labeled Compounds and Radiopharmaceuticals, 52: S142 (2009), each of which is herein incorporated by reference for its teaching of azides, phosphines, and the Staudinger ligation. In other embodiments, the substrate comprises the azide group, wherein the azide group can be transferred to an active-site amino acid residue (e.g., an active-site cysteine) of the AGT upon contact of the substrate with the AGT, wherein the radiolabeled probe comprises the phosphine group. In still further embodiments, the substrate further comprises a detectable label. The detectable label and the radiolabel are preferably suitable for imaging.

Thus, by way of a non-limiting example, in one embodiment, a BG comprising a phosphine group attached, directly or indirectly, thereto at the O(6)-position is provided, wherein the phosphine group of the BG is capable of in vivo conjugation with an azide group of a radiolabeled imaging probe. Accordingly, in some embodiments, once the BG reaches the target cell (e.g., a cancer cell) and contacts the ATase whereby at least the phosphine group is transferred to the active-site amino acid residue of the ATase, the radiolabeled imaging probe can target the ATase through conjugation of its azide group with the phosphine group of the BG via the Staudinger ligation.

In another embodiment, a BG is functionalized with an azide group that can react covalently via a Staudinger ligation with a phosphine probe comprising an imaging label. In some embodiments, the phosphine probe has the general formula (V):

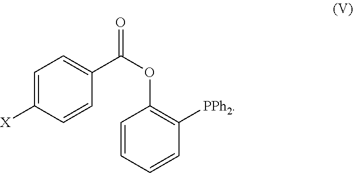

wherein X is a halogen atom, a radiohalogen, or a radiometal complexed to a chelating group. In other embodiments, the phosphine probe has the general formula (VI):

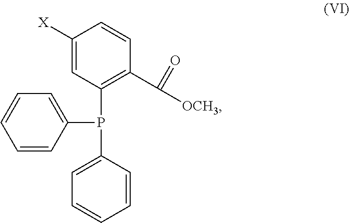

wherein X is a halogen atom, a radiohalogen, or a radiometal complexed to a chelating group.

### III. Compositions

In other aspects, a composition comprising one or more of the compounds, substrates, and/or the probes of the present invention is provided.

In one embodiment, the composition is a pharmaceutical composition suitable for administration to a subject. The subject can be any subject including a subject that may or may not be afflicted with a cancer. In some embodiments, the subject is a mammal, for example a human or a non-human. Other examples of mammals include, but are not limited to, dogs, cats, mice, rats, guinea pigs, horses, gorillas, chimpanzees, baboons, pigs, cows, and monkeys.

For example, the compounds, substrates, and/or probes of the present invention can be administered to a subject in combination with a physiologically acceptable carrier or excipient. For example, the compounds, substrates, and/or probes, optionally with the addition of a pharmaceutically acceptable carrier, can be formulated in an aqueous medium, with the resulting solution or suspension then being administered or sterilized prior to administration. Pharmaceutically acceptable carriers include, but are not limited to, physiologically compatible buffers such as Hanks' solution, Ringer's solution, dextrose, physiologically buffered saline, and water.

Compositions for injectable use include aqueous or non-aqueous injection solutions that may, optionally, contain various co-ingredients such as surfactants, anti-oxidants, buffers, bacteriostats, metal chelators (e.g., EDTA, EGTA), and solutes that render the composition isotonic with the blood of the intended subject; and aqueous and non-aqueous sterile suspensions which may include suspending agents and thickening agents. In some embodiments, injection solutions, dispersions, and suspensions can be prepared from sterile powders, granules, and tablets.

For example, pharmaceutical compositions suitable for parenteral administration can be prepared as solutions or suspensions of the compounds, substrates, and/or probes of the present invention in water (e.g., sterile water for injection) suitably mixed with a surfactant such as hydroxypropylcellulose. Dispersions can also be prepared in glycerol, liquid polyethylene glycols, and mixtures thereof in oils.

Compositions for oral administration can be liquid, semi-solid, or solid. Oral liquid preparations can be in the form of, for example, aqueous or oily suspensions, solutions, emulsions, syrups or elixirs, or may be presented as a dry product for reconstitution with water or other suitable vehicle before use. Oral liquid preparations can contain suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; water; non-aqueous vehicles (which may include edible oils), for example almond oil, oily esters such as glycerine, propylene glycol, or ethyl alcohol; preservatives, for example methyl or propyl p-hydroxybenzoate or sorbic acid; flavoring agents, preservatives, coloring agents and the like may also be used.

In some embodiments, a pharmaceutical composition can be presented in unit dose forms containing a predetermined amount of the compounds, substrates, and/or probes of the present invention per dose. For example, in one embodiment, the compositions comprises at least about 0.1% by weight, illustratively, about 0.1 to about 90%, about 1 to about 85%, about 10 to about 80%, about 20 to about 70%, about 30 to about 60%, and about 40 to about 50%, by weight, of the compounds, substrates, and/or probes of the present invention.

### IV. Methods

In other aspects, methods are provided that utilize the compounds, substrates, and/or probes of the present invention. For example, the compounds, substrates, and/or probes of the present invention can be used for one or more applications and/or methods including, but not limited to, labeling an ATase (e.g., AGT), determining treatment regimens, determining the effect of a DNA damaging agent, screening assays, and diagnostic and prognostic determinations.

The term “DNA damaging agent,” as used herein, is intended to be broad and includes, without limitation, chemotherapeutics, radiation (e.g., radiotherapy, ultraviolet radiation), heat, mutagenic chemicals (e.g., intercalating agents), toxins, viruses, reactive oxygen species, and cellular replication errors.

In some embodiments, the compounds, substrates, and/or probes are administered to a subject. Administering, which can be a single or multiple administration, can be by any appropriate route, including, but not limited to, parenteral, oral, intravenous, intramuscular, subcutaneous, intraarterial, intrathecal, intraventricular, transdermal, epidural, intraperitoneal, intranasal, topical, sublingual, rectal means, and combinations thereof.

Optionally, the composition can be injected directly into a tumor or into an organ in which a tumor is located. If desired, multiple administrations can be performed. Examples of tumors include, but are not limited to, gliomas, glioblastomas, astrocytomas, medulloblastomas, Hodgkin's tumors, and tumors of the colon, breast, ovary, prostate, kidney, uterus, pancreas, lung, testis, and muscle.

The amount of the compound to be administered can be determined empirically, and can depend on one or more factors such as, but not limited to, the route of administration, the size of the subject, and/or the type of cell expressing the ATase.

In one embodiment, the methods of the present invention comprise determining ATase levels. In some embodiments, determining comprises quantifying ATase levels. In other embodiments, ATase levels are quantified using PET or SPECT.

### 1. Preparing the Compound

In one embodiment, the present invention provides a method for preparing a compound comprising a substrate for an ATase, the method comprising:

performing a click reaction between an O⁶-benzylguanine (BG) having an azide functional group with a polypeptide having an alkyne functional group whereby the substrate is coupled to a polypeptide.

In some embodiments, the azide functional group is an azido-hexyloxymethyl group, wherein the polypeptide is a heptynoyl-modified peptide. In other embodiments, the heptynoyl-modified peptide comprises the amino acid sequence PKKKRKV (SEQ ID NO:1).

In another embodiment, the present invention provide a method for preparing a compound comprising a substrate for an ATase, the method comprising:

(a) conjugating an O⁶-benzylguanine (BG) having an active ester group with a polypeptide having an amine functional group, wherein the active ester group is capable of undergoing a reaction with the amine functional group to form an amide linkage.

In some embodiments, the reaction occurs in vivo by strain-promoted cycloaddition.

### 2. Labeling ATase

In one embodiment, the present invention provides a method for labeling an ATase (e.g., AGT) with a detectable label. The method comprises contacting a compound with the ATase, wherein the compound comprises a substrate for the ATase, wherein the substrate is coupled to a polypeptide, wherein the substrate is labeled with a detectable label bound to a chemical substituent of the substrate. In another embodiment, the substrate is an O⁶-benzylguanine (BG) having a radiolabel, wherein the polypeptide comprises an NLS.

In some embodiments, contacting occurs in vivo. For example, a composition comprising the compound can be administered to a subject for in vivo labeling of AGT.

In one embodiment, the present invention provides a method of detecting AGT in a subject, the method comprising:

(a) contacting the AGT of the subject with an O⁶-derivatized guanine compound comprising at the exocyclic O⁶ position a radiolabeled alkyl or benzyl group covalently coupled to a polypeptide under conditions whereby the radiolabeled alkyl or benzyl group is transferred from the O⁶-derivatized guanine compound to the AGT to form a radiolabeled AGT molecule; and

(b) detecting the radiolabeled AGT molecule.

In other embodiments, the present invention provides a method for in vivo labeling an ATase (e.g., AGT) in a subject. The method comprises administering to the subject a non-labeled substrate for an ATase, wherein the substrate has a reporting group that is bioorthogonal to a group of a labeled probe.

In one embodiment, the method further comprises administering to the subject the labeled probe.

In some embodiments, the non-labeled substrate is administered to the subject before administration of the labeled probe. In other embodiments, the non-labeled substrate and the labeled probe are contemporaneously administered to the subject. In one embodiment, a composition comprising the substrate and the labeled probe is administered to the subject.

In another embodiment, the non-labeled substrate is a BG derivative comprising a cyclooctyne group, wherein the labeled probe comprises an azide group, wherein the azide group of the labeled probe can form a triazole with the cyclooctyne group of the BG at physiological conditions via strain-promoted cycloaddition.

In some embodiments, the labeled probe molecule is ¹⁸F-labeled AZT or labeled DAPI.

In other embodiments, the non-labeled substrate has the formula (IV).

### 3. Determining a Treatment

In another embodiment, the present invention provides a method for determining a treatment regimen for a subject, the method comprising:

determining the subject's ATase levels, wherein determining comprises contacting an ATase of the subject with a compound comprising a substrate for an ATase, wherein the substrate is coupled to a polypeptide, wherein the substrate is labeled with a detectable label bound to a chemical substituent of the substrate, wherein the subject's ATase levels determine the treatment regimen.

The treatment regimen can be any prophylactic and/or therapeutic regimen suitable for preventing, treating, or delaying the onset of cancer. In some embodiments, the treatment regimen comprises chemotherapy, radiotherapy, or both.

In other embodiments, determining further comprises quantifying ATase levels. In one embodiment, ATase levels are quantified using PET or SPECT.

Without being held to any particular theory, it is believed that the effectiveness of a chemotherapeutic agent (e.g., alkylator chemotherapeutic agent) can be diminished if a tumor to be treated has AGT in amounts considerably higher than a threshold level. Several preclinical and clinical studies have established an inverse correlation between survival and AGT levels in a tumor. Alkylator chemotherapeutic agents such as temozolomide (TMZ) and carmustine (BCNU) can be used for the treatment of cancers of the brain as well as other types of malignancies, however, drug resistance can be a major impediment in alkylator chemotherapy. Thus, by assessing the presence or quantity of an ATase (e.g., AGT) in a subject, an appropriate treatment regimen for the subject, or which is predicted to have a greater degree of success, can be determined.

In one embodiment, the contacting occurs in vivo. For example, in some embodiments, the subject is administered a composition comprising the compound. For example, a compound comprising a radiolabeled BG analogue coupled to a polypeptide (e.g., NLS, MRT) can be administered to the subject and the radiolabeled group that is transferred from the labeled BG analogue to AGT can be qualitatively or quantitatively determined using any suitable technique such as, for example, scintigraphic imaging using standard nuclear medicine imagining equipment. In some embodiments, imaging can be performed repeatedly and provide spatio-temporal assessment of a tumor, for example. In one embodiment, a labeled ATase (e.g., labeled AGT) is determined using positron emission tomography (PET) or single photon emission tomography (SPECT).

For imaging, the amount of the compound administered can be determined empirically. In some embodiments, the compound is administered to the subject in an amount sufficient to yield the desired contrast with the particular imaging technique. For example, in some embodiments, wherein the label is a radionuclide, dosages of at least about 0.01 mCi, illustratively, about 0.01 to about 100 mCi, about 0.1 to about 50 mCi can be sufficient per about 60 to about 80 kg bodyweight.

In other embodiments, the treatment regimen comprises a chemotherapeutic regimen. In another embodiment, the chemotherapeutic regimen comprises administration of an alkylator. In some embodiments, the subject's ATase levels determine whether or not the therapeutic regimen should be initiated or continued.

In some embodiments, the present invention provides a method for determining a treatment regimen for a subject. The method comprises administering to the subject a non-labeled substrate for an ATase, wherein the substrate has a reporting group that is bioorthogonal to a group of a labeled probe.

In one embodiment, the method further comprises administering to the subject the labeled probe.

In other embodiments, the method further comprises determining the subject's ATase levels, wherein the subject's ATase levels determine the treatment regimen.

In some embodiments, the non-labeled substrate is administered to the subject before administration of the labeled probe. In other embodiments, the non-labeled substrate and the labeled probe are contemporaneously administered to the subject. In one embodiment, a composition comprising the substrate and the labeled probe is administered to the subject.

In another embodiment, the non-labeled substrate is a BG derivative comprising a cyclooctyne group, wherein the labeled probe comprises an azide group, wherein the azide group of the labeled probe can form a triazole with the cyclooctyne group of the BG at physiological conditions via strain-promoted cycloaddition.

In some embodiments, the labeled probe molecule is ¹⁸F-labeled AZT or labeled DAPI.

In other embodiments, the non-labeled substrate has the formula (IV).

### 4. Determining the Effect of a DNA Damaging Agent

In some embodiments, the present invention provides a method for determining the effect of a DNA damaging agent on the amount of AGT molecules in a tumor in a subject, the method comprising: determining the amount of AGT molecules in the tumor before, after, or contemporaneously with exposure of the tumor to the DNA damaging agent, wherein determining comprises:

(a) contacting the AGT of the subject with an O⁶-derivatized guanine compound comprising at the exocyclic O⁶ position a radiolabeled alkyl or benzyl group covalently coupled to a polypeptide under conditions whereby the radiolabeled alkyl or benzyl group is transferred from the O⁶-derivatized guanine compound to the AGT to form a radiolabeled AGT molecule.

In one embodiment, the method further comprises determining the amount of radiolabeled AGT molecules in the tumor, wherein the amount or the change in the amount before and after treatment is indicative of the effect of the DNA damaging agent. For example, in some embodiments, the method can be used to monitor the effect of exposure to the DNA damaging agent.

In other embodiments, the present invention provides a method for determining the effect of a DNA damaging agent on the amount of AGT molecules in a tumor in a subject. The method comprises administering to the subject a non-labeled substrate for an ATase before, after, or contemporaneously with exposure of the tumor to the DNA damaging agent, wherein the substrate has a reporting group that is bioorthogonal to a group of a labeled probe.

In one embodiment, the method further comprises administering to the subject the labeled probe.

In some embodiments, the non-labeled substrate is administered to the subject before administration of the labeled probe. In other embodiments, the non-labeled substrate and the labeled probe are contemporaneously administered to the subject. In one embodiment, a composition comprising the substrate and the probe is administered to the subject.

In another embodiment, the non-labeled substrate is a BG derivative comprising a cyclooctyne group, wherein the labeled probe comprises an azide group, wherein the azide group of the labeled probe can form a triazole with the cyclooctyne group of the BG at physiological conditions via strain-promoted cycloaddition.

In some embodiments, the labeled probe molecule is ¹⁸F-labeled AZT or labeled DAPI.

In other embodiments, the non-labeled substrate has the formula (IV).

### 5. Screening

In other embodiments, the present invention provides screening methods to screen for a molecule to identify candidate molecules that reduce or inhibit the expression and/or biological function/activity of an ATase (e.g., AGT). A candidate molecule may be capable of reducing the in vivo expression of an ATase, and/or interacting with the ATase to inhibit either the biological activity/function of the ATase or an interaction between the ATase and its in vivo modulator. In some embodiments, the molecule to be screened is from a small molecule library (e.g., a peptide or a non-peptide based library).

In some embodiments, an appropriate animal model is used to identify candidate molecules and/or determine the effect of exposure of a cell to such molecules.

In one embodiment, the method comprises:

determining a subject's ATase levels, wherein the subject is administered a candidate molecule, wherein determining comprises contacting an ATase of the subject with a compound comprising a substrate for an ATase, wherein the substrate is coupled to a polypeptide, wherein the substrate is labeled with a detectable label bound to a chemical substituent of the substrate, wherein ATase levels are indicative of reduction or inhibition of expression and/or biological function/activity of the ATase by the candidate molecule.

In some embodiments, the compound is administered to the subject before, after, or contemporaneously with administration of the candidate molecule. In one embodiment, the compound is administered to the subject simultaneously with administration of the candidate molecule, for example by way of administration of a composition comprising the compound and the candidate molecule.

For example, in one embodiment, a test agent can be administered to a mammal having (or suspected of having) a tumor and the level of labeled ATase (e.g., AGT) in the tumor in response to the test compound can be determined. A test agent that decreases the level of labeled ATase molecules in the tumor determines the test compound as a good substrate/modulating agent for ATase. In some embodiments, the test agent decreases the level of labeled ATase (e.g. labeled AGT) molecules in a tumor by at least 10%, illustratively, by about 10 to about 100%, about 20 to about 95%, about 30 to about 90%, about 40 to about 85%, about 50 to about 80%, and about 60 to about 70%.

If desired, radiolabeled AGT can be detected at multiple time points and/or after multiple administrations or concentrations of the test agent.

The tumor can be either experimentally induced or naturally occurring. A variety of tumor models suitable for use in this method are well known in the art, such as the murine colon 26-B carcinoma tumor model, the B 16 mouse melanoma model, athymic mice bearing a D341MED human brain tumor xenograft, or nude mice injected with HT29 colon tumor cells, A172 glioblastoma cells, or human brain tumor cell lines such as SF767 and U251 MG.

In other embodiments, the present invention provides a method for screening for a molecule to identify candidate molecules that reduce or inhibit the expression and/or biological function/activity of an ATase of a subject. The method comprises administering to the subject a non-labeled substrate for an ATase, wherein the substrate has a reporting group that is bioorthogonal to a group of a labeled probe.

In one embodiment, the method further comprises administering to the subject the labeled probe.

In other embodiments, the method further comprises determining the subject's ATase levels, wherein ATase levels are indicative of the efficacy of the subject's treatment.

The non-labeled substrate can be administered to the subject before or contemporaneously with administration of the labeled probe each. The non-labeled substrate and/or the labeled probe each can be administered to the subject before, after, or contemporaneously with administration of the candidate molecule.

In another embodiment, the non-labeled substrate is a BG derivative comprising a cyclooctyne group, wherein the labeled probe comprises an azide group, wherein the azide group of the labeled probe can form a triazole with the cyclooctyne group of the BG at physiological conditions via strain-promoted cycloaddition.

In some embodiments, the labeled probe molecule is “F-labeled AZT or labeled DAPI.

In other embodiments, the non-labeled substrate has the formula (IV).

This invention further pertains to novel agents identified by the screening assays of the present invention. Accordingly, it is within the scope of this invention to further use an agent identified by the screening method as described herein in treating cancer or in an appropriate animal model to determine its efficacy in treating cancer.

### VII. Kits

In other aspects, a kit is provided, wherein the kit comprises the compounds, substrates, and/or probes of the present invention. In some embodiments, the kit can comprise, in one or more suitable containers, a pharmaceutically acceptable formulation of at least one compound in accordance with the present invention. The kits may also contain other pharmaceutically acceptable formulations suitable for one or more applications including diagnosis, imaging, and/or therapy. For example, such kits may further comprise one or more chemotherapeutic or radiotherapeutic drugs; anti-angiogenic agents; anti-tumor cell antibodies; and/or anti-tumor vasculature or anti-tumor stroma immunotoxins or coaguligands; and test or candidate agents thereof.

The kits may have a single container that contains the compound of the present invention, with or without any additional components, or they may have distinct containers for each desired agent.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container. For example, wherein the components of the kit are in lyophilized form, the kit may optionally contain a sterile and physiologically acceptable reconstitution medium such as water, saline, buffered saline, and the like.

In some embodiments, the containers of the kit can include at least one vial, test tube, flask, bottle, syringe or other containers, into which the compounds of the present invention, and any other desired agent, may be placed and, preferably, suitably aliquoted. Where separate components are included, the kit will also generally contain at least a second container into which these are placed, enabling the administration of separated designed doses. The kits may also comprise additional containers for containing a sterile, pharmaceutically acceptable buffer or other diluent.

The kits may also contain devices by which to administer the compounds of the present invention, for example one or more needles or syringes, or even an eye dropper, pipette, or other such like apparatus. The kits of the present invention will also typically include structures for containing the vials, or such like, and other component, in close confinement for commercial sale, such as, e.g., injection or blow-molded plastic containers into which the desired vials and other apparatus are placed and retained.

In some embodiments, wherein a radioactive, or certain other labels, is involved, the user can, optionally, carry out the labeling reaction with the radionuclide in the clinical hospital, physician's office, or laboratory. For this purpose, or other purposes, the various reaction ingredients can then be offered to the user in the form of a kit. The kit is preferably designed so that the manipulations necessary to perform the desired reaction enable the user to prepare from the kit the desired composition by using the facilities that may be available to the user. For example, reagents useful in reactions to radiolabel the compound with a radionuclide and to conjugate to a polypeptide also may be included. Such kits also may comprise reagents for purifying the radiolabeled compound coupled to the polypeptide from the reaction mixture, as well as specific instructions for producing the radiolabeled compound coupled to the polypeptide using the kit components. Therefore the invention also relates to a kit for preparing a compound or composition according to this invention.

The kit to be supplied to the user may also comprise the ingredients) defined above, together with instructions for use. While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The following examples are given only to illustrate the present process and are not given to limit the invention. One skilled in the art will appreciate that the examples given only illustrate that which is claimed and that the present invention is only limited in scope by the appended claims.

### EXAMPLES

### Example 1

### Nuclear Localization Conjugate

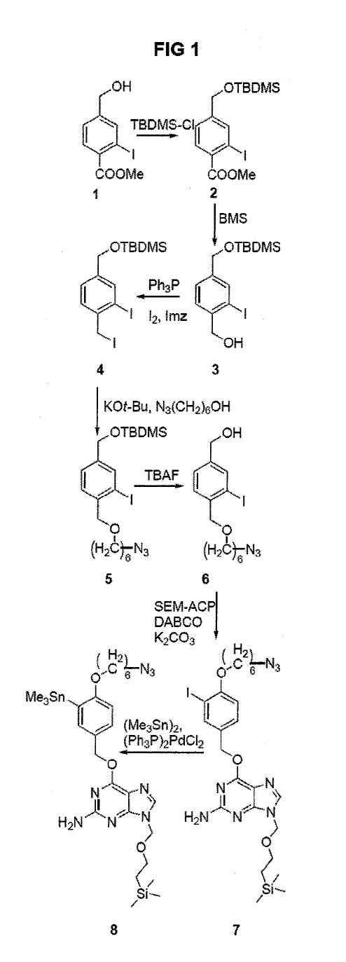

BG was modified with an azide function and the NLS peptide had an alkyne function and these two units were conjugated via click reaction. An IBG derivative appended with an azido-hexyloxymethyl group (AHMIB) was synthesized and conjugated to heptynoyl-modified PK₃RKV, a NLS derived from SV40 T-antigen (FIGS. 1 & 2). The radioiodinated analogue of AHOMIBG was derived from a tin precursor (62% radiochemical yield) and then conjugated to NLS using click reaction to obtain the final labeled product (55% conjugation yield).

Also, a BG derivative comprising an active ester group is coupled to an amine function in NLS. The peptide sequence is expanded to include negatively charged D- or L-amino acids (glutamates).

### Example 2

### NLS or MRT Conjugate

BG derivatives with an active ester can be used for conjugation to an NLS or MRT. Conjugation via click reaction also can be performed but modification of the NLS or MRT with an azide function is necessary, however, this can be accomplished.

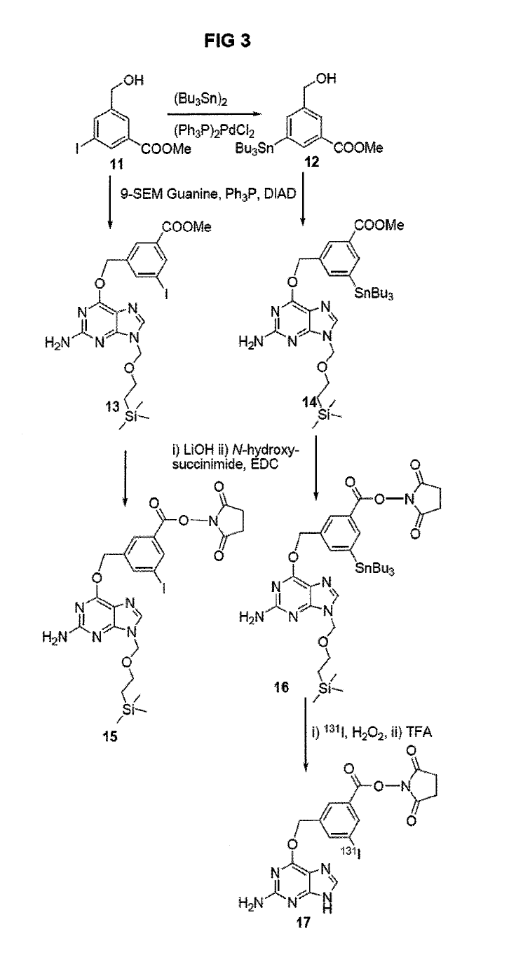

The scheme for the synthesis of an IBG derivative with an active ester group is shown in FIG. 3. Referring to FIG. 3, the synthesis of the iodo standard and the tin precursor attached with a cleavable group at the N-9 position (15 and 16) was accomplished. The tin precursor was radioiodinated to obtain [*I]15 in 45% radiochemical yield and the protecting group was removed with TFA to render the final product. The labeled molecule is then conjugated to the NLS or MRT.

### Example 3

### BG Derivative With Cyclooctyne Group

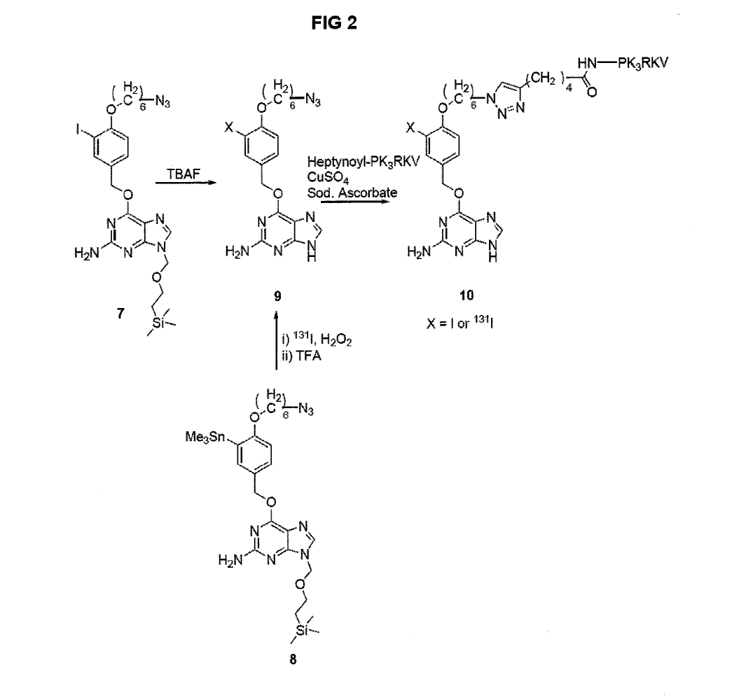

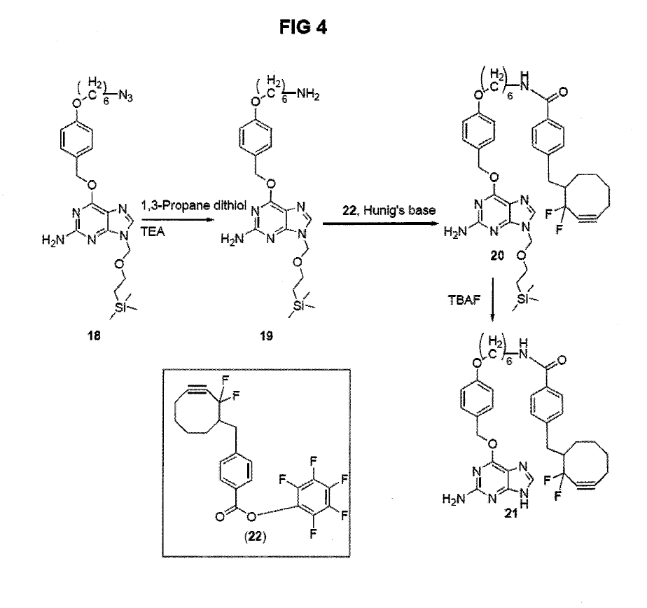

The scheme for the synthesis of a BG derivative with an attached cyclooctyne group is shown in FIG. 4. Compound 18, which can be synthesized following a procedure used for the preparation of 7 (FIG. 2), can be converted to 19 by treatment with 1,3-propane dithiol. Treatment of 19 with the DIFO derivative 22, derived from the corresponding acid (kindly provided by Carolyn Bertozzi, University of California, Berkeley) will yield 20. Deprotection of 20 with TBAF should yield the final compound 21. Other cyclooctyne derivatives reported recently also can be utilized for this purpose. AZT can be radioiodinated as reported or a derivative with a fluoroalkyl group at the 5-position can be synthesized for ¹⁸F-labeling.

### Example 4

### Materials and Methods

All chemicals were purchased from Aldrich Chemical Company except as noted. FBG (unlabeled) was obtained as a gift from Dr. Robert Moschel of National Cancer Institute-Frederick Cancer Research and Development Center, Frederick, Md. Sodium [¹³¹I]iodide in 0.1 N NaOH was supplied by DuPont-New England Nuclear (North Billerica, Mass.). [¹⁸F]Fluoride activity was produced on the Duke University Medical Center CS-30 cyclotron via the ¹⁸O (p,n) ¹⁸F reaction by irradiating [¹⁸O] H₂O in a small volume (300 μl) silver target. The activity was delivered to a solution of Kryptofix (10 mg in 1 ml CH₃CN) and potassium carbonate (1 mg in 5 μl water) in a glass tube and then evaporated with argon in an oil bath at 80° C. The dried activity was resolubilized in 50-100 μl of dry DMSO.

Melting points were determined on a Hake Buehler apparatus and were uncorrected. High pressure liquid chromatography (HPLC) was performed using one of three systems: 1) Two LKB Model 2150 pumps, an LKB Model 2152 control system, an LKB Model 2138 fixed-wavelength UV detector and a Beckman Model 170 radioisotope detector. Data were acquired using CIO-DAS08/JR internal and CIO-MINI137 external boards and Labtech DataLab/WIN software; 2) a Beckman System Gold HPLC equipped with a Model 126 programmable solvent module, a Model 168 diode array detector, a Model 170 radioisotope detector, and a Model 406 analog interface module; or 3) a Perkin-Elmer Series 4 Liquid Chromatograph connected to a Perkin-Elmer LC-95 UV/visible spectrophotometer detector and a Perkin-Elmer LCI-100 Laboratory Computing Integrator. Methods were programmed using a Perkin-Elmer 6312 display terminal.

For reversed-phase chromatography, a Waters μ Bondapak C18 (10 μm, 3.9×300 mm) column or a 3.9×300 mm Bondclone 10 (10μ) C-18 column (Phenomenex, Torrance, Calif.) was used. Normal-phase HPLC was performed using a 4.6×250 mm Partisil (10μ) (Alltech, Deerfield, Ill.).

Analytical TLC was performed on aluminum-backed sheets (Silica gel 60 F₂₅₄), and normal-phase column chromatography was performed using Silica gel 60, both obtained from EM Science (Gibbstown, N.J.). Column chromatographic fractions were collected using a Gilson model 203 micro fraction collector (Middleton, Wis.) or an ISCO Foxy 200 fraction collector (Lincoln, Nebr.). Products identified by TLC. In some cases, an ISCO UA-6 UV-VIS detector was placed between the column outlet and the fraction collector to identify fractions.

Preparative thick layer chromatography was performed using 20×20 cm, 1000μ plates (Whatman, Clifton, N.J.). Before applying the sample, the plates were run in ethyl acetate to clean the plates of any absorbed impurities. Radio-TLC was initially analyzed using a System 200 Imaging Scanner (BioScan, Washington, D.C.) and then cut into strips and counted using an automatic gamma counter (LKB 1282, Wallac, Finland). NMR spectra (¹H-300 MHZ and ¹³C-75 MHZ) were obtained on a Varian Mercury 300 spectrometer. Mass spectra were obtained on a Hewlett-Packard GC/MS/DS Model HP-5988A instrument, or on JEOL SX-102 high resolution mass spectrometer. Elemental analyses were provided by Galbraith Laboratories (Knoxville, Tenn.)

### Example 5

### Preparation of 6-(4-[¹⁸F]Fluoro-benzyloxy)-9H-purin-2-ylamine ([¹⁸F]FBG)

Para-[¹⁸F]fluorobenzaldehyde was prepared following previously described procedures (see, e.g., Vaidyanathan et al., Bioconjugate Chem., 5:352-356 (1994)). Briefly, 50-100 mCi of [¹⁸F]fluoride were resolubilized in DMSO (50-100 μl), added to 1-2 mg of 4-formyl-(N N,N-trimethyl)anilinium trifluoromethane sulfonate in a 5-ml Reacti® vial. The mixture was heated in an oil bath at 150° C. for 10 minutes. The cooled reaction mixture was diluted with water and passed through an activated C18 solid-phase cartridge (Waters). The cartridge was further washed with water (5-10 ml), and 4-[¹¹⁸F]fluorobenzaldehyde was finally eluted with methylene chloride (1-2 ml). The methylene chloride solution was dried with sodium sulfate and passed through a silica cartridge to remove any polar byproducts. Methylene chloride was removed using a rotary evaporator until a volume of about 0.1 ml was reached. Residual solution was transferred to a 1-dram vial. Use of argon to evaporate methylene chloride results in substantial loss of activity. Even with a rotary evaporator, it is important that the evaporation is stopped before the mixture dries.

DIBAL (0.1 ml, 1M solution in methylene chloride) was added to the above activity and the capped vial was left at room temperature for 10 minutes. After evaporating the methylene chloride, 1 ml of water was added carefully to the residual activity followed by 50-100 μl concentrated HC1. The resultant 4-[¹⁸F]fluorobenzyl alcohol was extracted from this solution with 3×1 ml of ethyl acetate. The combined ethyl acetate fractions were dried with sodium sulfate and concentrated on the rotary evaporator.

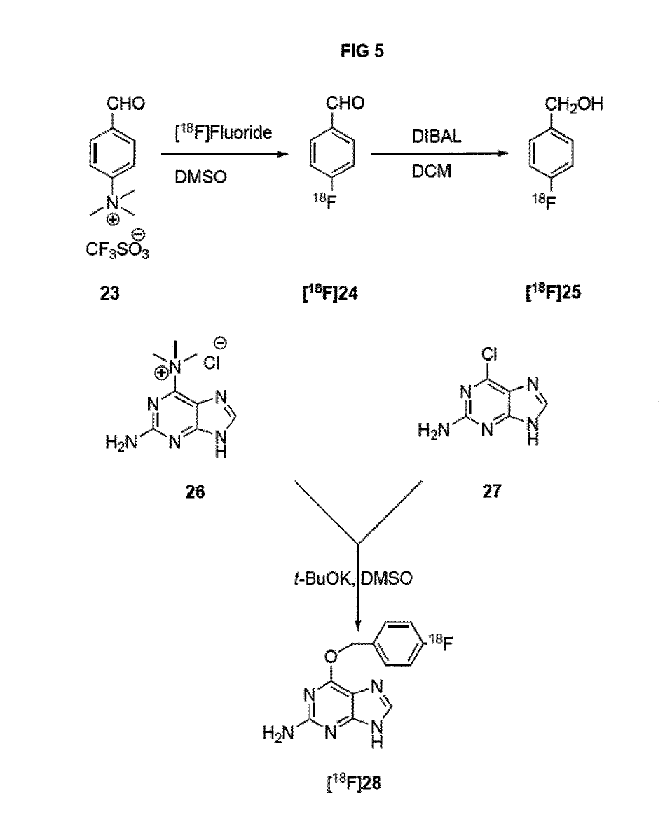

The above solution was transferred to a 1-ml Reacti® vial and evaporated to dryness. Potassium tert-butoxide (50 μl; 1M in THF) was added to this dried activity, and THF was evaporated under an argon flow. The residue was resolubilized in dry DMSO (Pierce, Rockford, Ill.; 50 μl) and the capped vial was heated at 100° C. for 5 minutes. Referring to FIG. 5, the quaternary salt 26 (1-2 mg) was added to the above solution, and the mixture was heated for an additional 10 minutes at 100° C. [¹⁸F]FBG ([¹⁸F]28; t_{R}=18-20 minutes) was isolated from this reaction mixture by reversed-phase HPLC (system 1) using the Bondclone column eluted with 0.1% TFA in 15:85 acetonitrile:water at a flow rate of 1 ml/min. The HPLC fractions containing [¹⁸F]28 were evaporated with argon to remove most of the acetonitrile, diluted with water, and passed through a solid-phase cartridge (tC18 ENV Sep Pak; Waters) activated with ethanol and water. The cartridge was washed with 5 ml of 0.9% saline, and the activity was eluted with 0.25 ml portions of ethanol. Most of the activity elutes in fractions 3-5. These pooled ethanol fractions were concentrated. For in vitro assays, the activity was reconstituted in Tris-buffer.

### Example 6

### Preparation of 6-(3-Iodo-benzyloxy)-9H-purin-2-ylamine (3-IBG)

Sodium hydride (60% in mineral oil; 80 mg, 2 mmol) was added to a solution of 3-iodobenzyl alcohol (498 mg, 2.13 mmol) in dry DMSO (0.5 ml), and the resultant yellow solution was stirred at room temperature for 1 hour. The quaternary salt 26 (FIG. 5) (229 mg, 1 mmol) was added to the mixture, and the stirring was continued for another 2 hours. Acetic acid (170 μl) was added to the clear, homogeneous reaction mixture, which was then added with vigorous stirring to about 30 ml of ether. The resultant precipitate was filtered, washed with ether and then with water, and dried over P₂O₅ under vacuum to give 218 mg (60%) of 7:mp 118-120° C. ; HPLC (System 3): Bondapak column was eluted with 0.1% TFA in 30:70 acetonitrile:water at 1 ml/min ; t_{R}=6.0 minutes as compared to 8-9 minutes for 3-iodobenzyl alcohol; ¹H-NMR (DMSO-d₆) 5.46 (s, 2H, benzylic), 6.28 (br s, 2H, NH₂), 7.21 (t, 1H, m-ArH ; J_{H-H}=8.5), 7.52 (d, 1H, o-ArH; J_{H-H}=8.5), 7.72 (d, 1H, p-ArH ; J_{H-H}=8.5), 7.84 (s, 1H, o′-ArH), 7.87 (s, 1H, H-8), 12.45 (br s, 1H, NH); ¹³C-NMR (CD₃OD) 67.77 (benzylic), 97.40 (ArC—I), 128.37 (guanine C-5 and o-ArCH), 131.19 (o′-ArCH and p-ArCH), 137.99 (m-ArCH and ipso-ArC), 138.07 (guanine CH-8 and C-6), 140.34 (guanine C-2 and C-4); MS (FAB⁺) m/z: 368 (MH⁺). HRMS calculated for C₁₂H₁₁IN₅O (MH+) m/z: 368.0008. Found: 368.0013±0.0009. Analyzed C₁₂H₁₀IN₅O C, H, N: calculated, C, 39.26; H, 2.75; N, 19.08. Found, C, 39.03; H, 2.87, N, 18.17.

### Example 7

### Preparation of 6-(3-Iodo-benzyloxy)-9-(2-trimethylsilanyl-ethoxymethyl)-9H-purin-2-ylamine (SEM-3-IBG)

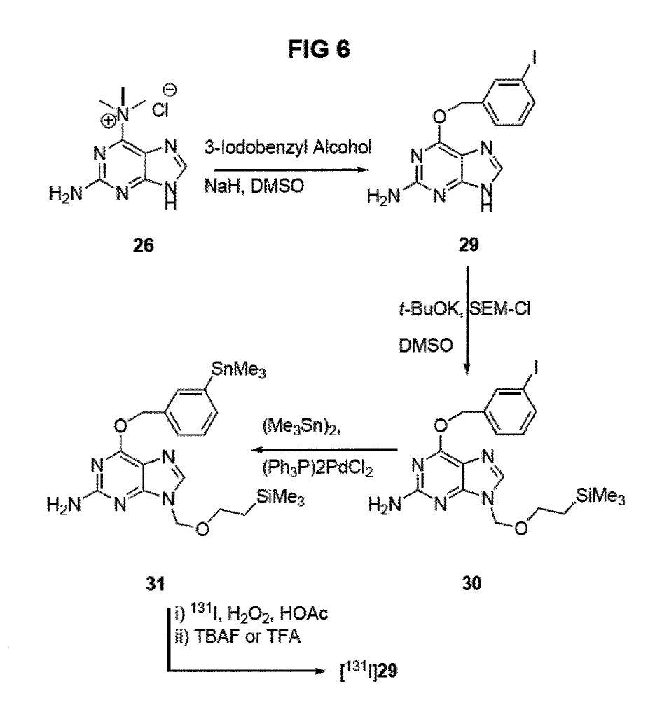

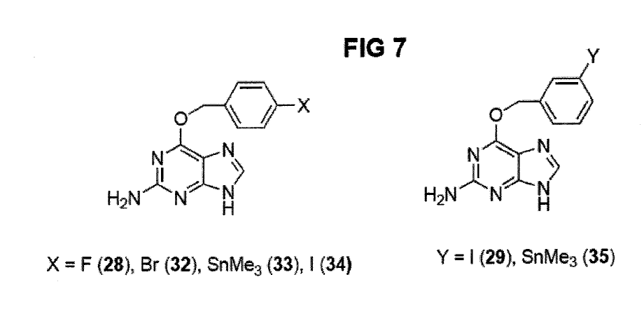

THF was evaporated from a 1M solution of potassium tert-butoxide in THF (0.83 ml, 830 mmol). An equivalent amount of sodium hydride was used with similar results. To the residue was added 29 (FIG. 6) (300 mg, 0.82 mmol) followed by DMF (silylation grade; Pierce, 4.2 ml). The mixture was stirred at room temperature under argon for 1 hour, cooled to 0° C., and SEM-chloride (138 mg, 0.83 mmol) was added drop-wise. The reaction was allowed to proceed overnight and was worked up by partitioning between ethyl acetate and water. Silica gel chromatography using 40:60 ethyl acetate:hexane afforded 105 mg (26%) of 30 (FIG. 7) as an oil/low melting solid: ¹H-NMR (CDCI₃)-0.02 (s, 9H, —SiMe₃), 0.93 (m, 2H,—Si—CH₂—), 3.58 (m, 2H, Si—CH₂—CH₂—O—), 4.83 (br s, 2H, NH₂), 5.44 (s, 2H, benzylic), 5.50 (s, 2H,—N—CH₂—O—), 7.09 (t, 1H, m-ArH ; J_{H—H}=7.7), 7.48 (d, 1H, o-ArH ; J_{H—H}=7.5), 7.65 (d, 1H, p-ArH; J_{H—H}=7.8), 7.74 (s, 1H, H-8), 7.86 (s, 1H, o′-ArH), ¹³C-NMR (CDCl₃);-2.05 (—SiMe₃), 17.06 (—CH₂—SiMe₃), 66.17 (—Si—CH₂—CH₂—O—), 66.28 (benzylic), 71.14 (—N—CH₂—O—), 93.41 (C—I), 112.48 (guanine C-5), 126.57 (o-ArCH), 129.29 (m-ArCH), 136.20 (o′-and p-ArCH), 137.86 (ArCH-ipso), 138.72 (guanine CH-8), 153.66 (guanine C-6), 158.52 (guanine C-2), 159.87 (guanine C-4); FIRMS (FAB⁺) calculated for C₁₈H₂₅IN₅O₂Si (MH⁺) m/z: 498.0820+0.0018. Analyzed C₁₈H₂₄IN₅O₂Si C, H, N: calculated C, 43.46; H, 4.86; N, 14.08. Found, C, 43.56; H, 4.69, N, 13.84.

### Example 8

### Preparation of 9-(2-Trimethylsilanyl-ethoxymethyl)-6-(3-trimethylstannanyl-benzyloxy)-9H-purin-2-ylamine

A mixture of 30 (FIG. 7) (50 mg, 0.1 mmol), hexamethylditin (210 mg, 0.64 mmol) and dichlorobis(triphenylphospine)palladium(II) (35 mg, 0.05 mmol) in 3 ml of dioxane was heated at reflux under argon for 1 hour. Dioxane was removed from the dark reaction mixture, and the residue was resuspended in ethyl acetate. The suspension was filtered through a bed of Celite, and the bed was washed thoroughly with ethyl acetate. The filtrate was concentrated to obtain an oil. This oil was applied to a bed of silica gel, and very non-polar impurities were removed by eluting with hexane. The required product and other byproducts were isolated from the silica gel bed by eluting with ethyl acetate. Ethyl acetate was removed from this solution. The residual oil was subjected to preparative TLC using 40:60 ethyl acetate:hexane to obtain 39 mg (73%) of 31 (FIG. 7) as a white solid: mp 135-136° C., ¹H-NMR (CDCl₃)-0.03 (s, 9H,—SiMe₃), 0.29 (s, 9H,—SnMe₃), 0.92 (m, 2H, —CH₂—SiMe₃), 3.57 (m, 2H,—Si—CH₂—CH₂—O—), 4.87 (br s, NH₂), 5.43 (s, 2H, benzylic), 5.55 (s, 2H,—N—CH₂—O—), 7.31-7.61 (m, 4H, ArH), 7.72 (s, 1H, H-8), ¹³C-NMR (CDC1₃)-9.37 (—SiMe₃), -1.32 (—SnMe₃) 17.80 (—CH₂—SiMe₃), 66.87 (—Si—CH₂—CH₂—O—), 68.47 (benzylic), 71.84 (—N—CH₂—O—), 127.90 (guanine C-5), 128.28 (o-ArCH), 128.56 (m-ArCH), 129.24 (o′-and p-ArCH), 135.47 (ArCH-ipso), 135.60 (C—Sn), 135.94 (guanine CH-8), 139.27 (guanine C-6), 154.28 (guanine C-2), 159.31 (guanine C-4); MS (FAB⁺) calculated for C₂₁H₃₃IN₅O₂Si¹²⁰ Sn (M⁺) m/z: 535.1429. Found: 535.1429±0.0001. Analyzed C₂₁H₃₃IN₅O₂SiSn C, H, N: calculated, C, 47.21; H, 6.23; N, 13.11. Found, C, 47.35; H, 6.09, N, 12.89.

### Example 9

### Preparation of 6-(3-[¹³¹I]Iodo-benzyloxy)-9H-purin-2-ylamine(34¹³¹I]IBG)

### A. Two-Step Approach

Referring to FIG. 6, to a ½-dram vial containing 0.2 mg of 31 was added 1-2 μl of ¹³¹I in 0.1N NaOH (about 1 mCi), followed by 10 μl of a 3:1 (v/v) solution of acetic acid:30% H₂O₂. The mixture was sonicated for about 30 seconds, injected onto a normal-phase HPLC column, and eluted with 0.2% acetic acid in 50:50 ethyl acetate:hexane at a flow rate of 1 ml/minute. The required product [¹³¹I]30 (t_{R}=˜13 minutes) was isolated in more than 90% radiochemical yield.

Solvents from HPLC fractions containing [¹³¹I]30 were evaporated to a small volume, transferred to a ½-dram vial. The solvents were again evaporated to dryness. The residual radioactivity was treated with trifluoroacetic acid (50 μl) for 5 minutes at room temperature. Most of the trifluoroacetic acid was evaporated with an argon flow and triturated with 50 μl of ethyl acetate twice to insure its complete removal. Methanolic ammonia (50 ul) was added to the vial. The vial was vortexed, and methanol and ammonia were evaporated off under a flow of argon. The radioactivity was reconstituted in methanol and injected onto a normal-phase HPLC column eluted with 0.1% acetic acid in ethyl acetate at a flow rate of 1 ml/min. The desired [¹³¹I]29 (t_{R}=˜13 minutes) was isolated in 55-60% radiochemical yield. Under these HPLC conditions, neither 3-iodobenzyl alcohol nor 30 were retained in the column. To ensure the authenticity of the final product, it was subjected to TLC in 5% (v/v) methanol in ethyl acetate along with the unlabeled standard. The retention factor for 29 was 0.3, while those for 3-iodobenzyl alcohol and 30 were 0.8,0.9, respectively.

The deprotection of [¹³¹I]30 to [¹³¹I]29 also was accomplished with tetrabutylammonium fluoride (TBAF) in THF. For this, TBAF (50 μl, 1 M in THF) was added to a vial containing the residue of [¹³¹I]30, and the capped vial was heated at 65-70° C. for 25 minutes. THF was evaporated. The activity was reconstituted in a mixture of 25 μl methanol and 5 μl acetic acid and injected on to a normal-phase HPLC as described above. The radiochemical yield was 85-90%.

### B. Single-Step Approach

After radioiodination of 30, solvents were removed by co-evaporating twice with a few microliters of benzene. The residual activity was heated with 50 μl of TBAF in THF for 25 minutes at 65-70° C. After evaporating THF, the activity was processed as above, and [¹³¹I]7 was isolated by normal-phase HPLC in 70% radiochemical yield.

### Example 10

### Cell Culture and Transfection

CHO-K1 cells were maintained in αMEM (Gibco, Grand Island, N.Y.) containing 10% fetal bovine serum. CHO cells were transfected with a plasmid expressing human AGT using FuGENE (Roche Molecular Biochemicals) according to the manufacturer's protocol for transfection of adherent cells. After 48 hours, geneticin (Gibco) was added at a concentration of 500 μg/ml. Clones were isolated from individual cell foci.

### Example 11

### AGT Activity Assays

Inactivation of cellular AGT was measured by adding varying concentrations of FBG or IBG to cell cultures that had reached 80-90% confluence. After 4 hours of drug treatment, cell extracts were prepared. AGT activity was measured by assaying the loss of [³H]—O⁶-methylguanine from a [³H] methylated calf thymus DNA substrate as described by, e.g., Dolan et al., PNAS, 87:5368-5372 (1990). The results are expressed as the percentage of the AGT activity present in cell cultures that were not treated with FBG or IBG. For each concentration, the assay was performed in duplicate.

### Example 12

### In Vitro Binding of FBG or IBG by AGT

Purified human AGT with a (His)₆ tag was generously provided by Dr. Anthony E. Pegg of Hershey Medical Center, Pennsylvania State University. About 50,000 counts of [¹⁸F]FBG or [¹³¹I]IBG were added in the presence or absence of increasing amounts of cold FBG or IBG, respectively, to 10 μg AGT or to a control for nonspecific binding, BSA. The incubation carried out in 0.1 ml 50 mM Tris-Cl, pH 7.5, 5 mM DTT, 0.1 mM EDTA in the presence of 10 μg calf thymus DNA. After incubation for 30 minutes at 37° C., the protein was precipitated by the addition of 200 μg carrier BSA and 1 ml cold 12% TCA. The precipitated proteins were collected on GF-C (Whatman) filters, which were washed extensively with 5% TCA. The results are expressed as the percentage of input activity retained on the filter. For each concentration, the assay was performed in triplicate. The assay was performed twice for both [¹⁸F]FBG and [¹³¹I]IBG.

### Example 13

### Depletion of AGT Activity by FBG and IBG

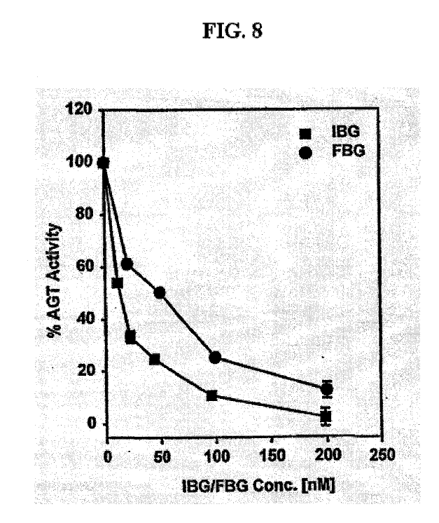

Because IBG is a new compound, its ability to deplete AGT was determined using CHO cells transfected with pCMV-AGT to express AGT. In parallel, the ability of FBG to deplete AGT under the same conditions also was measured. As shown in FIG. 8, IBG depleted AGT from these cells to a degree higher than that depleted by FBG. An IC₅₀ of 50 nM was measured for FBG, similar to the value reported for FBG in HT29 human colon tumor cells. On the other hand, IBG had an IC₅₀ of 15 nM, suggesting that it is more potent than FBG in depleting AGT. These results demonstrate that introduction of an iodine at the meta-position of the benzyl group in BG did not result in a loss of AGT-depleting ability.

### Example 14

### Binding of Radiolabeled FBG and IBG to Purified AGT

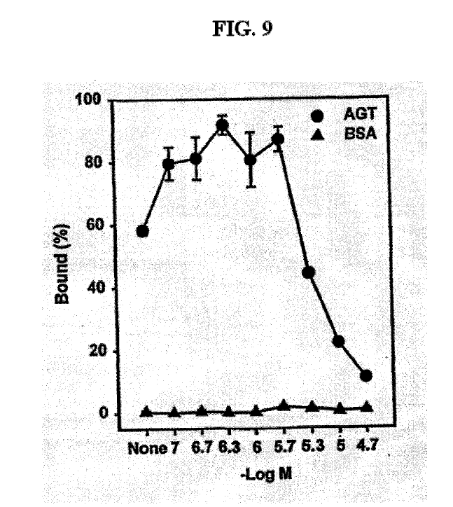

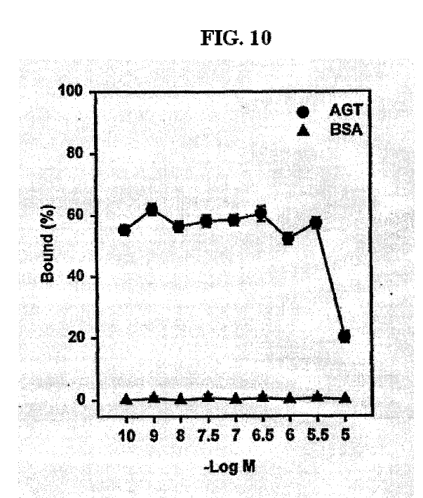

To investigate whether AGT recognizes radiolabeled FBG and IBG as substrates, the efficacy of transfer of their radiolabeled benzyl groups to AGT was determined using the purified protein. For [¹⁸F]FBG, the percent of input activity that bound to AGT increased with increasing concentrations of unlabeled FBG reaching a maximum of about 90% at 0.5 μM of FBG (FIG. 9). Saturation was observed at concentrations higher than 2 μM. An IC₅₀ of 5 μM was calculated. About 60% of radiolabeled IBG bound to AGT over a wide range of concentrations (FIG. 10) with saturation noticed at concentrations higher than 3 μM. The IC₅₀ for IBG was about 6 μM. These relatively high IC₅₀ values further suggest that higher specific activity will not be a requirement for these radiopharmaceuticals. In both cases, the binding was specific as demonstrated by the fact that very little radioactivity was bound to the nonspecific protein BSA with maximum observed AGT to BSA binding ratios of 661 and 35 for [¹⁸F] FBG and [¹³¹I]FBG, respectively. These results are qualitatively similar to that obtained with O⁶-(p-hydroxy [³H]methylbenzyl) guanine, the only other BG derivative labeled at the benzyl part of the molecule. The preparation of ¹³C-and ¹⁴C-labeled O⁶-benzylguanosines has been reported by, e.g., Madelmont et al., J. Lab. Comp. Radiopharm., 31:793-800 (1992).

Although IBG depletes AGT from cells to a greater degree than FBG, [¹⁸F]FBG had a higher binding to the purified protein than [¹³¹I]IBG. This may be due to differential transmembrane transport of the two compounds as a result of differences in their lipophilicity. The lipophilicities of IBG and FBG were not determined per se. IBG is expected to be more lipophilic. Reversed-phase HPLC has been used to determine lipophilicities. In comparison to FBG, a higher percentage of acetonitrile was needed to elute IBG from a revered-phase column, suggesting that IBG is more lipophilic. However, it should be pointed out that the more non-polar 4-fluorobenzyl alcohol elutes earlier (t_{R}=10-11 minutes) than FBG (t_{R}=18-20 minutes) under identical reversed-phase HPLC conditions.

A prerequisite for insertion of ¹⁸F onto a benzene ring by nucleophilic substitution is that the ring contains a suitable leaving group, such as-NO₂ or a quaternary ammonium triflate, that is positioned ortho or para to a strongly electron withdrawing group such as NO₂ (22,23). It may be possible to prepare an FBG precursor, such as the one with an-NO₂ or a quaternary ammonium group in the place of fluorine, from which [¹⁸F]FBG may be produced in a single step. However, this chemistry will not be facile due to the lack of a suitably placed electron withdrawing group in the molecule. Referring to FIG. 5 (scheme 1), to circumvent this, it was necessary to first prepare ¹⁸F-labeled 25 and couple it to the guanine skeleton. Although the chemistry of [¹⁸F]25 has been reported, a slightly different set of conditions was used for its preparation. Para-[¹⁸F] fluorobenzaldehyde, [¹⁸F]24, was prepared as described in Vaidyanathan et al., Bioconjugate Chem., 5:352 (1994). Reduction of [¹⁸F]24 was initially accomplished with NaBH₄ in ethanol at 80° C. However, this involved removal of the initial solvent methylene chloride from a solution of [¹⁸F]24 and reconstitution of [¹⁸F]24 in ethanol. In addition to the extra time needed, this process resulted in a considerable loss of activity due to the very volatile nature of [¹⁸F]24. It was found that a commercially available solution of DIBAL in methylene chloride was very effective in converting [¹⁸F]2 to [¹⁸F]25 at room temperature. Preparation of [¹⁸F]25 from [¹⁸F] fluoride could be accomplished in about an hour in an overall decay-corrected radiochemical yield of 50%.

Unlabeled FBG was originally prepared by the treatment of 2-amino-6-chloropurine (ACP) at 100-130° C. for over 24 hours with an excess of the sodium salt of 4-fluorobenzyl alcohol in 4-fluorobenzyl alcohol as solvent. These conditions are not adaptable to ¹⁸F labeling. Even if one started with Curie-quantities of [¹⁸925 and microgram amounts of ACP, the concentration of 25 would be substantially sub-stoichiometric. In addition, the long reaction time is not suitable with ¹⁸F. This chemistry was initially attempted by conducting the reaction in a solvent such as THF or DME without success. A number of O⁶-hetarylguanines have been prepared in excellent yields based on an earlier report using 26, instead of ACP, as the starting material. These reactions proceeded under mild conditions and involved easy work-up. The procedure essentially involves the treatment of 26 with the pre-formed sodium alcoholate (2 equivalents of sodium hydride and 5.6 equivalents of desired alcohol) in DMSO at room temperature for 1 hour. Treatment of the pre-formed sodium salt of 4-fluorobenzyl alcohol (using sodium hydride) with an equimolar quantity of 26 in DMSO at room temperature resulted in the formation of 28 in more than 80% yield (HPLC) in 30 minutes. It was also possible to convert ACP to 28 by doing the reaction in DMSO, albeit at a higher temperature. Treatment of ACP with 2 equivalents of the potassium salt of 4-fluorobenzyl alcohol (using potassium tert-butoxide) in DMSO at room temperature did not result in the formation of 6. However, heating the reaction mixture at 130-140° C. for 30 minutes resulted in about 40% conversion, determined by HPLC, of ACP to 28.

A higher reaction temperature was used to augment the rate of the radiochemical reaction of [¹⁸F] 25 with 26. Typically, [¹⁸F] 25 was heated with a base in DMSO for 5 minutes at 100° C. (to ensure deprotonation), and heated again for 10 minutes after the addition of 26. Sodium hydride was initially used as a base, but owing to cumbersome handling of sodium hydride, a readily available and easily measurable solution of potassium tert-butoxide in THF was subsequently used, giving equally good results. Although in some cases decay-corrected radiochemical yields of about 70% have been obtained for this coupling reaction, the yield generally was around 40%. In a few reactions, ACP was substituted for 26; however, the decay-corrected radiochemical yield was only about 25%. Little or no radioactivity corresponding to [¹⁸F] 25 eluted in reversed-phase HPLC. The remainder of the radioactivity was retained in the column, suggesting the possibility of highly non-polar byproduct(s). About 7-9 mCi of C⁸F]28 was obtained starting from 100 mCi of resolubilized [‘BFJfluoride in about 2 hours. Although the specific activity of the final product has not been systematically determined, no co-eluting carrier peak was generally observed on HPLC. Nonetheless, unlike receptor-based radiopharmaceuticals, very high specific activity is not necessary for O⁶-alkyl guanine derivatives and may even be counterproductive because transfer of alkyl groups by AGT follows second order kinetics.

One of the most commonly used techniques for the preparation of radioiodinated compounds is the radioiododestannylation of the corresponding tin precursor. Initially, we envisaged the preparation of 31 (FIG. 7) from the reported compound 30. It may be possible to prepare 32 or its radioiodinated analogue by the halodestannylation of 31; however, attempts to convert 30 to 31 by treatment with hexamethylditin and bis-triphenylphosphine palladium dichloride in dioxane were unsuccessful. This was probably due to the insolubility of 30 in dioxane.

We prepared several ACP surrogates with protecting groups, such as trimethylsilylethyl, trimethylsilylethyoxymethyl (SEM), tert-butyloxymethyl, and pivaloyloxymethyl, at the N′-position, and converted some of these to their O′-(substituted) benzyl derivatives. Recently, SEM and p-methoxyphenylmethyl (MPM) protecting groups were used to facilitate the coupling of 6-halopurine to glycosylamines.

Although it should have been possible to convert N9-SEM-ACP to 30 (FIG. 6), we used an alternative approach following the procedure of McElhinney et al., J. Med. Chem., 41:5265-5271 (1998). Referring to FIG. 6, (scheme 2), compound 29 was prepared from 26 and commercially available 3-iodobenzyl alcohol in 60% isolated yield and converted to 30 by treatment with sodium hydride or potassium tert-butoxide, and SEM-C1. The unoptimized isolated yield of 30 from this reaction was only 26%. In contrast, yields of 50-85% have been reported for modification of amines with a SEM Group. The N7-regioisomer is a byproduct in the N-alkylation of guanines ; however, the N′IN7 ratio generally is very high. We did not attempt to isolate the NV7-regioisomer of 8 if any was formed. Compound 30 was smoothly converted to 31 using the palladium-catalyzed stannylation.

Radioiodination of 31 to [¹³¹I]30 was performed easily by sonication for 30 seconds with ¹³¹I and a mixture of acetic acid and hydrogen peroxide. The radiochemical yield was more than 90%. Tetrabutylammonium fluoride (TBAF) and TFA are among reagents that have been used for the removal of the SEM group. We initially used TFA for converting [¹³¹I] 30 to [¹³¹I]29 based on preliminary results for the deprotection of N-(trimethylsilylethyl) ACP (TMSE-ACP) to ACP. TMSE-ACP was treated with TBAF in DMF or DMSO at room temperature and at 60° C. TMSE-ACP remained intact at room temperature for at least one hour. Analysis of the reaction mixtures left at room temperature for 20 hours and those heated at 60° C. for 5 minutes by HPLC showed several by-product peaks. On the other hand, a single product peak corresponding to ACP was observed when TMSE-ACP was treated with TFA for 5 minutes at room temperature. Treatment of [¹³¹I] 30 with TFA at room temperature for 5 minutes gave 55-60% radiochemical yield of [¹³¹I]29; 15-20% of unreacted [¹³¹I] 30 was recovered. Conducting the reaction for a longer time did not improve the radiochemical yield.

A combination of radio-TLC, and normal-phase and reversed-phase HPLC was used to verify that the final product was indeed IBG and not the 3-iodobenzyl alcohol. Very little if any of 3-iodobenzyl alcohol was detected in the reaction mixture. In addition, the final product bound to AGT specifically (vide infra), which further supports its authenticity. It was surprising that TFA treatment did not cleave the ethereal linkage between guanine and the benzyl moieties because the instability of BG to acidic conditions has been reported. This may be due to the lack of water in the reaction mixture or a rate factor may be involved. Both IBG and FBG were stable during reversed-phase chromatography involving 0.1% TFA in water (pH 2) as a co-solvent.

No deprotection was observed when [¹³¹I] 30 was treated with a 1 M solution of TBAF in THF at room temperature up to 30 minutes, and [¹³¹I] 30 was recovered quantitatively. Increasing the reaction temperature to 70° C. resulted in the formation of [¹³¹I] 29 in a time-dependent fashion. A maximum radiochemical yield of 85% was obtained after about 25 minutes, at which time essentially only one radioactive peak corresponding to IBG was seen on HPLC. This is contrary to the results described above for the deprotection of TMSE-ACP with TBAF at macromolar levels.

The above procedures involve two normal-phase HPLC separations. Although it was possible to perform the two steps in the same pot consecutively, there was an unlabeled peak co-eluting with the desired radioactive peak when the final mixture was subjected to normal-phase HPLC. This was presumably 33 (FIG. 7), because the same peak was seen when 31 (FIG. 6) was treated with TBAF and injected onto the HPLC. Use of reversed-phase HPLC may be necessary to separate 29 (FIGS. 6) and 33 (FIG. 7). However, isolating [¹³¹I]29 (FIG. 6) from reversed-phase HPLC fractions involves a solid-phase extraction and removal of a solvent such as ethanol or methanol. Because it is much easier to evaporate ethyl acetate, the two normal-phase HPLC procedure is preferred. Preparation of 33 (FIG. 7) in sufficient quantities should permit direct conversion of 33 (FIG. 7) to [¹³¹I]29 FIG. 6) in a single step.

### Example 15

### Binding of BG Derivatives of the Present Invention to AGT

Whenever appropriate, these assays are performed in a paired-label format, for example a new agent labeled with ¹³¹I will be paired with [¹²⁵I]IBG/[¹²⁵I]IBdG. Labeled analogues will be evaluated both before and after conjugation with NLS peptides.

Purified human AGT with a (His)₆ tag is used for these studies. About 50,000 counts of a labeled compound is added in the absence or presence of increasing amounts of unlabeled BG to ˜10 μg AGT or, as a control for nonspecific binding, to BSA in 0.1 ml 50 mM Tris-Cl, pH 7.5, containing 5 mM DTT, 0.1 mM EDTA and 10 μg calf thymus DNA. While calf thymus DNA is necessary for certain 9-substituted BG derivatives, its presence can be deleterious in the inactivation of AGT by others. For this reason, parallel assays are performed in which DNA is replaced by hemocyanin. For unconjugated IBdG analogues, after incubation for 30 min at 37° C., the protein is precipitated by the addition of 200 μg carrier BSA and 1 ml cold 12% TCA. The precipitated proteins are collected on GF-C filters which are washed extensively with 5% TCA. The percentage of input activity that is retained on the filter is calculated. For each BG concentration, the assay is performed in triplicate, and the entire assay is done twice. The concentration of the BG needed to reduce the binding of the tracer (i.e., a labeled BG derivative) to 50% of the maximum (IC₅₀) is calculated and from this the relative potency of the novel tracer to bind to AGT is inferred. In evaluating the NLS peptide conjugates of the present invention, in addition to above, SDS PAGE and HPLC (size-exclusion and reversed phase) also are used to characterize radioactive species present after exposure to AGT.

### Example 16

### Binding of BG Derivatives of the Present Invention to AGT in Human Cancer Cells

Cell binding assays are performed using a panel of commercially available human cancer cell lines including DAOY, TE-671, HT29, and D 283 Med (HCR), which are known to express a high level of AGT. For each assay, 100 nCi of a labeled BG derivative (i.e., tracer) of the present invention is incubated with 5×10⁵ cells in appropriate cell culture medium at 37° C. At the end of incubation period, cells are washed, lysed, and counted for radioactivity. The uptake is expressed as the percent of input counts associated with the cells. Nonspecific binding is evaluated using cells pretreated with a large excess of BG to block AGT.

To determine uptake and retention kinetics of the BG derivative, the cells are incubated with the tracer for various periods of time and the cell-associated radioactivity is determined as described above; from this, the time at which maximum binding occurs is obtained. To determine for how long the bound radioactivity remains associated with the cells, cells are allowed to take up the activity for the optimal time period. The medium containing the radioactivity is removed, and the cells are washed and then incubated with fresh medium for various time periods. Again, the cell-associated radioactivity is determined and plotted as a function of time.

To demonstrate a correlation between cellular uptake of the tracer and the AGT content of the cells, the AGT content of the cells is variably depleted by incubating the cells for 4 h with various concentrations of BG. Subsequently, the BG-containing medium is removed and the cells are incubated further for 2 h with 100 nCi of the tracer. The cell-associated radioactivity as a percent of the input counts is plotted against the BG concentration and correlation coefficients from the best fit of these plots is determined. An indirect measure of the AGT content of the cells treated with various amount of BG is obtained by conducting SDS PAGE of cell extracts from a similar assay. The bands corresponding to AGT in the gel are quantified using phosphor imaging.

### Example 17

### Bio-Distribution/Metabolism of BG Derivatives of the Present Invention in vivo

The BG derivatives of the present invention are further evaluated using xenograft mouse models. These experiments are performed using TE-671 and DAOY xenograft models implanted as subcutaneous, intracranial (i.e.) and neoplastic meningitis models. Tumor AGT content in these models are depleted completely or variably by administration of BG or dBG. Alternatively, xenografts generated from cell lines lacking AGT are utilized as negative controls. When two labeled analogues are compared, the studies are performed in a paired-label format. All animals are treated according to guidelines based on the Public Health Service Policy on Humane Care and Use of Laboratory Animals.

Bio-distribution in mice bearing subcutaneous xenografts: For a typical experiment, mice having xenografts of about 250-500 mm³ in size are injected via their tail vein with 5-10 μCi of a BG derivative (i.e., tracer). Groups of 5 animals are sacrificed by an overdose of isofluorane at time points between 15 min to 24 hr after injection of the tracer; time points greater than 24 hr also is included if warranted. Tumor, blood, and other major organs, especially those that are known to have high amounts of AGT such as liver and lungs are isolated, weighed, and the radioactivity in them is assessed. In comparing the tissue radioactivity levels of two agents, the statistical significance of the difference between the two values is determined using a paired or an unpaired t test depending on the type of study. Tumor tissue from groups of mice from a parallel study is harvested, extracts made and assayed for AGT, for example by SDS PAGE/phosphor imaging.

Bio-distribution in intracranial models: TE-671 and DAOY intracranial xenografts are established. Bio-distribution is performed essentially as described above for s.c. model, except that mice are injected i.v. with Evan's blue dye prior to sacrifice to guide in the dissection of i.c. tumors. A group of 3-5 mice are used for each time point. Mice are injected with 10-20 μCi of the radiolabeled compound and sacrificed at different time points from 15 min to 24 hr; longer time points are included if warranted. Tumor and organs such as liver, kidney, and spleen are isolated, and tissues that cannot be processed immediately after isolation are stored at −80° C. to minimize possible degradation of labeled entities. Tissues from the individual animals within a group are processed either separately or after combining them. Tissues are first counted for radioactivity, and homogenized with a hand-held homogenizer using 2×500 μl of PBS containing the protease inhibitors aprotinin (0.02 mg/ml), pepstatin (0.07 mg/ml), EDTA (5 mM), and PMSF (4 mM). The tissue homogenates are centrifuged, and the supernatants and pellets are counted for radioactivity. The supernatants are filtered using a 0.45-μm filter and the filtrate is analyzed by size-exclusion HPLC and SDS-PAGE as described below. To determine the low molecular weight catabolites, two volumes each of acetonitrile and NaOH (100 μM final) is added to a portion of the supernatants and centrifuged. The resultant supernatant is filtered further through a 5-kDa cut-off filter and the filtrate is analyzed by reversed-phase HPLC. In addition to the above tissues, blood and urine samples are collected at the same time points when tissues are collected. Methanol is added to urine samples to a final concentration of 35% (v/v) and the mixture is kept on ice for 2 hr. It is centrifuged at 14,000×g for 10 min, and the supernatant filtered through a 0.2-μm filter prior to reversed phase HPLC analysis. Blood is centrifuged to isolate serum. Size-exclusion HPLC and SDS PAGE analysis is performed to characterize protein-bound radioactivity. Two volumes each of acetonitrile and NaOH (100 μM final) is added, the mixture is centrifuged and the supernatant analyzed by reversed phase HPLC.

Size-exclusion HPLC is performed using a gel filtration HPLC column eluted with PBS at 1 ml/min. In addition to the protein molecular weight standards, the radiolabeled AGT analogue also is injected onto the HPLC to determine the approximate molecular weights of the high molecular weight species. Supernatants of tissue homogenates and the respective labeled AGT also are analyzed by SDS-PAGE using a 4-20% gradient gel under non-reducing conditions. The radioactivity in various bands is quantified using a phosphor image analysis system. The percent of total radioactivity in tumor and other tissues that is associated with intact tracer, or is bound to AGT or any other proteins is determined. Reversed-phase HPLC of non-protein bound radioactivity is performed to identify any low molecular weight catabolites.

### Example 18

### Determining Tumor Resistance or Sensitivity to Chemotherapy

To demonstrate whether tumors with AGT levels above and below a threshold, as determined by labeled NLS peptide conjugates (e.g., a compound having the formula III, wherein X is a radioisotope) uptake, are resistant and sensitive, respectively to a DNA damaging agent (e.g., radiation, chemotherapeutic (e.g., alkylator chemotherapy)), bio-distributions are conducted as detailed above using both xenograft models described earlier.

For example, tumors are variably depleted of AGT by pre-administration of graded amounts of BG (i.p. or intratumorally). Tumors from mice in parallel groups are harvested and their AGT content is determined. For alkylator therapy, other groups of mice treated with same amounts of BG are administered with BCNU (35 mg/m 2) or temozolomide (170 mg/m2) 2 hr after BG administration. Mice from this group are monitored up to 90 days to determine therapeutic response using tumor growth delay and regression as endpoints. Bio-distributions are conducted periodically in these mice, for example by microPET imaging, after administration of labeled NLS peptide conjugates. At the same time, tumor samples are obtained from these mice by fine needle aspiration biopsy and their AGT content is determined.

The ability to image and assess AGT levels avoids unnecessary therapies (e.g. chemotherapy, radiotherapy, etc.) and/or provides for personalizing therapy. In addition to the economic benefits, it can spare patients from the major side effects of certain therapies, e.g., alkylator chemotherapy, and allow them to be triaged earlier to alternative more effective treatments. Thus, the imaging tools and methods in accordance with the present invention provide powerful techniques for personalizing therapy, e.g. chemotherapy and/or radiotherapy, for individual patients.

### Example 19

### Stability of BG Derivatives of the Present Invention and Nuclear Localization

The in vitro serum stability of labeled compounds, before and after conjugating to NLS peptides, also is ascertained. The in vitro metabolism is determined with regard to effects mediated by live cells. These assays are performed in paired label format for direct comparison of two agents where appropriate.

Metabolism: These studies are performed using both tumor cells and serum. Cells are allowed to take up the tracer under optimal conditions, and after removal of the medium containing unbound radioactivity, cells are reincubated with fresh medium. At different time points, the supernatant is removed and saved for HPLC analysis. Cells are lysed by vortexing vigorously with 100 μl of 0.5% NP40 in PBS containing the protease inhibitors aprotinin (0.02 mg/ml), pepstatin (0.07 mg/ml), EDTA (5 mM), and PMSF (4 mM); the resultant mixture is incubated for 10 min at room temperature. Cell debris is removed by centrifugation. Cell culture supernatants and cell lysates after removal of debris is analyzed by size-exclusion HPLC and SDS PAGE to determine the amount of radioactivity that is associated with AGT or other proteins. Both cell culture supernatants and cell lysates are filtered through 5-kDa cut off filters to separate low molecular weight radioactivity from those bound to proteins. The amount of radioactivity in the low- and high molecular weight fractions is determined by counting the filtrate and the cartridge. Reversed-phase HPLC of filtrates obtained from both cell culture supernatants and lysates is performed to identify and quantify any radiolabeled low molecular weight catabolites and to determine the percent of total radioactivity that is associated with the intact tracer. Similar studies are performed using mouse serum. For this, the radiolabeled compounds is incubated with serum at 37° C. for various periods of time and then processed and analyzed by HPLC as described above.

Nuclear localization: In addition to the SDS PAGE, the extent of cell-bound radioactivity that is present in the cell nucleus is determined. Briefly, the cells (1×10⁷ per 5 ml media in a T-150 flask) are allowed to take up the radioactive compound under optimum conditions. Then the cells are pelleted, and re-suspended in 1 ml of cytoskeleton (CSK) buffer [0.5% Triton X-100, 300 mM sucrose, 100 mM NaCl, 1 mM EGTA, 2 mM MgCl₂, and 10 mM PIPES (pH 6.8)] and are incubated on ice for 2 min. The nuclear pellet is isolated by centrifugation at 560×g, washed with 1 ml of CSK buffer sans Triton-100 and counted in a gamma counter. Alternatively, the Nuclei EZ kit available from Sigma is utilized following the manufacture's protocol for isolating the nuclear fractions. The total cell-associated radioactivity is determined in parallel. If more than 75% of the radioactivity is not in the cell nuclei, sub-cellular fractionation is performed to determine the percent of cell-associated radioactivity in other organelles.

1. A compound comprising a substrate for an alkyltransferase (ATase), wherein the substrate is coupled to a polypeptide.

2. The compound of claim 1, wherein the substrate further comprises a label.

3-4. (canceled)

5. The compound of claim 1, wherein the ATase is an alkylguanine-DNA alkyl transferase (AGT) AGT.

6-9. (canceled)

10. The compound of claim 1 9, wherein the substrate is an O⁶-benzylguanine (BG).

11. The compound of claim 1, wherein the polypeptide comprises a nuclear localization sequence (NLS).

12. The compound of claim 1, wherein the polypeptide comprises the amino acid sequence PKKKRKV.

13. The compound of claim 1, wherein the polypeptide comprises an activatable cell-penetrating polypeptide (ACPP) sequence.

14. The compound of claim 1, wherein the polypeptide comprises a sequence corresponding to at least a portion of a cell-specific ligand.

15. (canceled)

16. The compound of claim 1 having the formula (I): 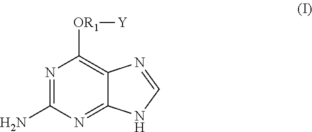
wherein R₁ is a benzyl group, wherein Y is a the polypeptide.

17-18. (canceled)

19. The compound of claim 1 having the formula (I): 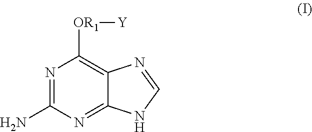
wherein R₁ is a benzyl group substituted at the ortho, meta, or para position with:
an azide functional group,
an azido-hexyloxymethyl group,
R₂R₃ where R₂ represents an alkyl of 1-4 carbon atoms and R₃ represents an azide functional group or an azido-hexyloxymethyl group,
R₄R₅ where R₄ represents carbonyl and R₅ represents succinimidyloxy, or
R₆R₇R8 where R₆ represents a hexyloxymethyl group, R₇ represents an amine, and R8 represents a cyclooctyne group; and
wherein Y is the polypeptide.

20. (canceled)

21. The compound of claim 1 having the formula (II): 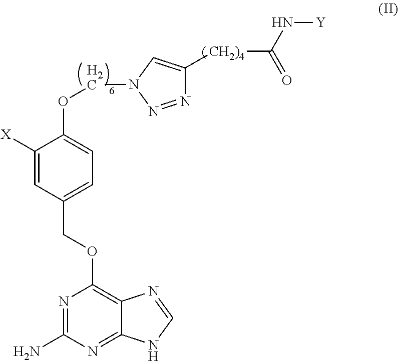
wherein X is a halogen atom or a radioisotope thereof, wherein Y is the polypeptide.

22. (canceled)

23. The compound of claim 1 having the formula (III): 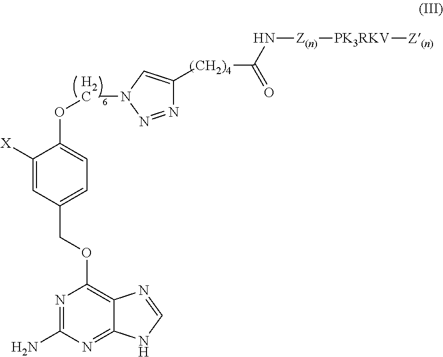
wherein X is a halogen atom or an radioisotope thereof, wherein Z and Z′ are each independently an amino acid, wherein n is an integer greater than or equal to zero.

24-33. (canceled)

34. A method for preparing he compound of claim 1, the method comprising:
performing a click reaction between an O⁶-benzylguanine (BG) having an azide functional group with a polypeptide having an alkyne functional group whereby the substrate is coupled to the polypeptide.

35-38. (canceled)

39. A composition comprising the compound of any one of claim 1, wherein the composition further comprises a pharmaceutically acceptable carrier.

40. A method for labeling an ATase, the method comprising:
contacting the compound of claims 1 with the ATase, wherein the substrate is labeled with a detectable label bound to a chemical substituent of the substrate.

41-51. (canceled)

52. A method of detecting an ATase in a subject, the method comprising:
(a) contacting the AGT of the subject with the compound of claim 1, wherein the compound further comprises at the exocyclic O⁶ position a radiolabeled alkyl or benzyl group covalently coupled to the polypeptide under conditions whereby the radiolabeled alkyl or benzyl group is transferred from the compound to the AGT to form a radiolabeled AGT molecule; and
(b) detecting the radiolabeled AGT molecule.

53-56. (canceled)

57. A method for determining a treatment regimen for a subject, the method comprising:
determining the subject's ATase levels, wherein determining comprises contacting an ATase of the subject with the compound of claim 1, wherein the substrate is labeled with a detectable label bound to a chemical substituent of the substrate, wherein the subject's ATase levels determine the treatment regimen.

58-66. (canceled)

67. A method for determining the effect of a DNA damaging agent on the amount of AGT molecules in a tumor in a subject, the method comprising:
determining the amount of AGT molecules in the tumor before, after, or contemporaneously with exposure of the tumor to the DNA damaging agent, wherein determining comprises:
(a) contacting the AGT of the subject with the compound of claim 1, wherein the compound comprises at the exocyclic O⁶ position a radiolabeled alkyl or benzyl group covalently coupled to the polypeptide under conditions whereby the radiolabeled alkyl or benzyl group is transferred from the O⁶-derivatized guanine compound to the AGT to form a radiolabeled AGT molecule; and
(b) determining the amount of radiolabeled AGT molecules in the tumor, wherein the amount is indicative of the effect of exposure to the DNA damaging agent.

68. A method for screening for a molecule to identify candidate molecules that reduce or inhibit the expression and/or biological function/activity of an ATase, the method comprising:
determining a subject's ATase levels, wherein the subject is administered a candidate molecule, wherein determining comprises contacting an ATase of the subject with the compound of claim 1, wherein the substrate is labeled with a detectable label bound to a chemical substituent of the substrate, wherein ATase levels are indicative of reduction or inhibition of expression and/or biological function/activity of the ATase by the candidate molecule.

69-74. (canceled)

75. A kit comprising the compound of claim 1 or a pharmaceutically acceptable formulation thereof.


SEQUENCE LISTING (21 sequences)

SEQ ID NO: 1 - protein, 7 aa, Artificial Sequence
  nuclear localization signal from SV40 virus large T antigen
PKKKRKV

SEQ ID NO: 2 - protein, 19 aa, Artificial Sequence
  nuclear localization signal from nculeoplasmin-based bipartate sequence
NLSKRPAAIKKAGQAKKKK

SEQ ID NO: 3 - protein, 9 aa, Artificial Sequence
  nuclear localization signal from c-myc-based sequences
PAAKRVKLD

SEQ ID NO: 4 - protein, 11 aa, Artificial Sequence
  nuclear localization signal from c-myc-based sequences
RQRRNELKRSF

SEQ ID NO: 5 - protein, 38 aa, Artificial Sequence
  nuclear localization signal from hRNPA1 M9-based sequence
NQSSNFGPMKGGNFGGRSSGPYGGGGQYFAKPRNQGGY

SEQ ID NO: 6 - protein, 41 aa, Artificial Sequence
  nuclear localization signal from importin-alpha IBB domain-based sequence
RMRKFKNKGKDTAELRRRRVEVSVELRKAKKDEQILKRRNV

SEQ ID NO: 7 - protein, 8 aa, Artificial Sequence
  nuclear localization signal from myoma T protein-based sequences
VSRKRPRP

SEQ ID NO: 8 - protein, 8 aa, Artificial Sequence
  nuclear localization signal from myoma T protein-based sequences
PPKKARED

SEQ ID NO: 9 - protein, 8 aa, Artificial Sequence
  nuclear localization signal from human p53-based sequence
PQPKKKPL

SEQ ID NO: 10 - protein, 12 aa, Artificial Sequence
  nuclear localization signal from mouse c-abl IV-based sequence
SALIKKKKKMAP

SEQ ID NO: 11 - protein, 5 aa, Artificial Sequence
  nuclear localization signal from influenza virus NS1
DRLRR

SEQ ID NO: 12 - protein, 7 aa, Artificial Sequence
  nuclear localization signal from influenza virus NS1
PKQKKRK

SEQ ID NO: 13 - protein, 10 aa, Artificial Sequence
  nuclear localization signal from hepatitis virus delta antigen
RKLKKKIKKL

SEQ ID NO: 14 - protein, 10 aa, Artificial Sequence
  nuclear localization signal from mouse Mx1-based sequence
REKKKFLKRR

SEQ ID NO: 15 - protein, 20 aa, Artificial Sequence
  nuclear localization signal from teh human poly (ADP-ribose) polymerase-based bipartate sequence
KRKGDEVDGVDEVAKKKSKK

SEQ ID NO: 16 - protein, 17 aa, Artificial Sequence
  nuclear localization signal from steroid hormone receptor-based sequence
RKCLQAGMNLEARKTKK

SEQ ID NO: 17 - protein, 23 aa, Artificial Sequence
  cell penetrating peptide (CPP) / polycationic sequence
EEEEEDDDDKAXRRRRRRRRRXC

SEQ ID NO: 18 - protein, 22 aa, Artificial Sequence
  cell penetrating peptide (CPP) / polycationic sequence
EEEEEDDDDKARRRRRRRRRXC

SEQ ID NO: 19 - protein, 19 aa, Artificial Sequence
  cell penetrating peptide (CPP) / polycationic sequence
EDDDDKAXRRRRRRRRRXC

SEQ ID NO: 20 - protein, 23 aa, Artificial Sequence
  cell penetrating peptide (CPP) / polycationic sequence
EEDDDDKARXRRXRRXRRXRRXC

SEQ ID NO: 21 - protein, 19 aa, Artificial Sequence
  cell penetrating peptide (CPP) / polycationic sequence
DDDDDDKARRRRRRRRRXC